(19) United States
(12) Patent Application Publication
(10) Pub. No.: US 2026/0182909 A1
(43) Pub. Date: Jul. 2, 2026

(54) SYSTEMS AND METHODS FOR POSITIVE AIRWAY PRESSURE (PAP) TREATMENT ADHERENCE OR SLEEP OPTIMIZATION

(71) Applicant: The University of Chicago, Chicago, IL (US)

(72) Inventors: Esra Tasali, Chicago, IL (US); Becky Tucker, Chicago, IL (US); Bonnie Hughes, Chicago, IL (US); Bonnie Spring, Chicago, IL (US); Angela F. Pfammatter, Chicago, IL (US)

(21) Appl. No.: 19/125,509

(22) PCT Filed: Oct. 31, 2023
(86) PCT No.: PCT/US2023/036473

§ 371 Date: Apr. 29, 2025

Related U.S. Application Data
(60) Provisional application No. 63/420,985, filed on Oct. 31, 2022.

(51) Int. Cl. A61B 5/00; A61M 16/06; G16H 50/30

(52) U.S. Cl. CPC A61B 5/4833; A61B 5/4815; A61M 16/06; G16H 50/30; A61M 2205/502; A61M 2230/63

(57) ABSTRACT

Disclosed herein are systems and methods for monitoring PAP treatment adherence. Systems and methods disclosed herein provide techniques to quantify PAP wear time in proportion to the time in bed or time asleep, and take into account individual factors affecting sleep patterns to provide feedback to patients and providers for motivating adherent use of the PAP device.

### CROSS-REFERENCE TO RELATED APPLICATION

This application claims the benefit of U.S. Provisional Patent Application No. 63/420,985, filed on Oct. 31, 2022, which is incorporated by reference herein in its entirety.

### STATEMENT OF GOVERNMENT INTEREST

This invention was made with Government support under Contract Nos. R01DK120312, R01DK125749, and R01DK136214 awarded by the National Institute of Diabetes and Digestive and Kidney Diseases of the National Institutes of Health. The Government has certain rights in the invention.

### TECHNICAL FIELD

The disclosure relates to systems and methods for monitoring positive airway pressure (PAP) treatment adherence and/or sleep optimization (e.g., extension of sleep duration), and providing subsequent personalized, actionable feedback for evaluating treatment adherence as related to individual sleep patterns.

### BACKGROUND

Sleep apnea, such as obstructive sleep apnea (OSA) and central sleep apnea, is a global public health and economic burden, estimated to affect one billion people in the world. Sleep apnea is a sleep disorder characterized by recurrent complete or partial upper airway obstruction resulting in reduced oxygen levels at night, sleep fragmentation, and poor sleep quality. Untreated sleep apnea is commonly associated with daytime sleepiness and neurocognitive impairment, increasing the risk of motor vehicle accidents. Furthermore, there is strong evidence showing that sleep apnea is associated with increased cardiometabolic risk and all-cause mortality. Currently, positive airway pressure (PAP) (e.g., such as continuous positive airway pressure, CPAP, bilevel positive airway pressure, BiPAP, or adaptive servo-ventilation, ASV), applied at night, is considered the treatment of choice for sleep apnea and there is no FDA-approved drug treatment for sleep apnea.

As an example, a PAP device uses a hose connected to a mask (e.g., patient interface) to deliver continuous air pressure which works as a “pneumatic splint” to prevent upper airway closures during sleep. PAP can be applied using a variety of masks (e.g., nasal masks, nosepieces such as nasal pillows, or full-face masks) worn on the face at night and is highly efficacious in treating sleep apnea. In at least some cases, effective sleep apnea treatment includes all-night PAP use (e.g., during the entire time spent in bed) to optimally treat obstructive respiratory events, hypoxia, and sleep fragmentation, and thus prevent adverse health effects that are associated with hours slept without wearing PAP.

If the PAP mask is removed from the patient's face, the device has no therapeutic effect on the condition, and sleep apnea resumes. Thus, the overall effectiveness of PAP therapy is linked to mask wear during the time spent in bed sleeping at night. At least some guidelines specify 4 hours of PAP use per night as a cut-off point defining adequate treatment adherence for all patients, which does not consider how much time each patient spent in bed at night for sleeping. Notably, individual sleep patterns can vary greatly within the same patient from night to night and varies greatly between patients depending on their age, sex, race/ethnicity, and other socioeconomic factors influencing their sleep habits. In clinical practice, according to Medicare criteria, patients who wear their PAP for 4 hours or more per night for 70% of the nights are considered “adherent” to therapy without any personalized tracking or assessment of sleep patterns.

In at least some cases, effective sleep apnea treatment includes all-night PAP use during the entire time spent in bed for optimally treating respiratory events and preventing adverse health effects associated with time spent without wearing PAP. However, common problems prevent users from wearing the PAP mask. These problems include a leaky mask, wrong mask size and fit, a stuffy nose, or a dry mouth. Additionally, some patients have trouble getting used to wearing a PAP mask, have difficulty learning to tolerate forced air, unintentionally remove the mask during sleep, and/or feel claustrophobic while wearing the mask. These issues affect the efficacy of PAP treatment and limit the amount of time a patient wears the device. Thus, a considerable portion of PAP users may fail to achieve an optimal all-night treatment. For example, as many as 46-83% of patients on PAP may be described as insufficiently treated based on current clinical adherence definitions.

For patients with suboptimal treatment adherence, the patient is advised clinically to increase their PAP usage (e.g., without any clear goal that is individualized to patient's needs or sleep patterns), which is often discouraging to the patient and challenging for the patient and provider. Further, health insurances may or may not cover PAP therapy according to the level of “adherence”. A patient who is deemed “non-adherent” to treatment may be required to give up the device. For example, Medicare covers a 3-month trial of PAP therapy after a patient has been diagnosed with sleep apnea. But after the trial period, Medicare may continue to cover PAP therapy only if the patient meets Medicare's “adherent” threshold. For those who are considered “adherent”, Medicare continues to pay the supplier to rent a PAP machine for about 13 months, after which the patient can own the machine.

Taken together, an accurate metric to define adherence to PAP treatment that takes into account the patient's individual sleep patterns is critical in sleep apnea management and has significant public health implications, and improved monitoring and assessment of adherence to treatment is needed in order to provide clinically meaningful treatment goals and appropriate recommendations/interventions to improve health outcomes.

Further, evidence suggests that sleeping less than seven hours per night on a regular basis is associated with adverse health consequences. For example, insufficient sleep duration has been increasingly recognized as an important risk factor for obesity. Prospective epidemiologic studies suggest that short sleep duration is an important risk factor for weight gain. Accordingly, there is a need to accurately track a patient's sleep patterns in order to aid in the patient's weight management and/or to improve the user's general health.

### SUMMARY

The disclosure provides systems and methods for providing PAP treatment adherence and monitoring. The disclosure recognizes that the current metric for determining who is adherent to PAP therapy is inadequate in at least some use cases, and provides a PAP use monitoring system that is personalized to individual sleep patterns and measures PAP mask wear time in proportion to objectively measured time spent in bed for sleeping. The disclosure also provides systems and methods for sleep optimization (e.g., extending sleep duration to a healthier length) in a population that may not be undergoing PAP treatment or that may or may not have sleep apnea.

In at least some implementations, “time in bed” can represent any rest time (e.g., naps on a chair, couch etc.) in addition to time spent in bed at night for sleeping. In some embodiments, systems and methods disclosed herein include a smartphone application (“app”) that provides meaningful feedback that aims to encourage and improve PAP adherence. This metric changes and improves the current clinical guidelines, as well as impacts the insurance companies' definition of who is “adherent” to therapy, thus resulting in widespread public health implications. In some embodiments, time in bed can refer to “recommended time in bed” or “recommended sleep duration,” as determined by a healthcare provider or by American Academy of Sleep Medicine based guidelines and/or recommendations (e.g., a minimum of seven hours of sleep being healthy for adults), as opposed to an individual's “actual” time in bed.

As noted above, poor adherence to PAP treatment is a common problem. As such, technology and methods are needed for improved PAP adherence tracking and for supporting patients and providers in their clinical decision making and treatment/management of disease. In at least some current adherence tracking systems (e.g., smartphone apps) simply capture the number of hours the PAP mask is worn per night, but do not account for the hours spent in bed without wearing the PAP device. The use of the 4-hour cut-off point defining adequate treatment adherence is arbitrary and could be misleading.

For example, based on a 4-hour PAP adherence threshold, a patient who uses PAP for 5 hours but spends 8 hours in bed per night would be considered adherent. Yet, the patient's true PAP adherence would be only 63% as a percentage of time spent in bed. In this scenario, the patient's treatment adherence will be considered “adherent” when it should be considered suboptimal. This results in the patient not being clinically advised to increase their PAP usage, when in actuality their non-adherence should be clinically addressed.

Embodiments of the disclosure solve this challenge by implementing more accurate PAP adherence goals (e.g., for individuals and/or health care providers) and more effective treatment/management guidelines using a system and techniques to quantify PAP wear time in proportion to objectively measured time spent in bed. Importantly, embodiments disclosed herein take into account the fact that sleep patterns can vary considerably from night to night and between individuals. In embodiments of the disclosure, these and other individual factors are taken into account and also include the time spent in bed for sleeping in the calculation of an accurate treatment adherence metric. Further, embodiments of the disclosure use this adherence calculation and integrated system to provide meaningful feedback to patients for motivating optimal use of the PAP device and achieving associated health goals. In some implementations, this adherence metric can be used to provide individualized treatment and health management for patients. Such health management can include, e.g., one or more lifestyle interventions to improve sleep hygiene or to implement sleep extension based on recommended healthy sleep duration guidelines, which in turn can be part of programs to prevent obesity, improve weight loss, and/or optimize cardiometabolic/cardiovascular health. Further, in some implementations, existing treatment and health management guidelines can be modified to reflect this adherence metric, such that they are more effective in improving the health of the patient.

In at least some implementations, a system tracks PAP wear relative to objectively assessed time in bed, represented as percent PAP adherence. To harvest the data needed to calculate percent PAP adherence, the embodiments of the disclosure leverage connected technologies, for example, in some embodiments, a PAP device to track PAP mask wear time and a wearable sensor (e.g., an activity tracker and/or a sleep tracker) to track time spent in bed. This PAP adherence metric is personalized to each patient's night-to-night variability in sleep patterns and provides markedly different information as compared to available PAP tracking technologies that simply report how many hours a PAP mask was worn without accounting for the time spent in bed without wearing PAP.

By leveraging wearable sensor technology that can objectively capture time in bed, embodiments of the disclosure provide more informative, clinically meaningful measures of PAP adherence that can be implemented into clinical guidelines. Because the systems and methods disclosed herein define PAP adherence metrics that considers together both PAP wear and time spent in bed and leverage integrated technology to capture the percent PAP adherence metric, the disclosure fulfills an important gap in sleep apnea management for patients and health care providers.

Further, because the system feeds back personalized information about PAP use relative to the time in bed, the disclosure supports patients in reaching their goal of 100% PAP adherence (e.g., wearing their PAP during the entire time they spend in bed for sleeping). The real-time app data on percent PAP adherence may be used not only as a self-management tool for patients but also as a more accurate treatment efficacy and personalized adherence monitoring tool for healthcare providers, who in turn can provide to the patient clinically meaningful recommendations/interventions to improve health outcomes.

Embodiments of the disclosure provide a technology platform that allows for a dashboard to display patient information (e.g., from an electronic device, a computer application, etc.) so that both the patient and provider can see progress towards goals that takes into account individual sleep-wake patterns. Additionally, the system integrates lifestyle behavior tracking features (e.g., diet, physical activity, body weight, sleep tracking, etc.) and thus enhances self-management and positive behavior change toward weight loss goals in patients (e.g., with sleep apnea or without sleep apnea). Obesity is a major risk factor for sleep apnea, and weight loss is recommended (but remains a major challenge) in this patient population. Even still, obesity is a major public health concern, and weight loss may be improved in the general population by sleep extension (e.g., sleeping at least 7 hours per night, ideally between 7 to 9 hours based on adult healthy sleep duration recommendations). Strong clinical evidence suggests that sleep deprivation (e.g., short sleep duration) is associated with overeating and weight gain. Without wishing to be limited by mechanism or theory, sleep extension may reduce energy intake, which in turn can result in negative energy balance (e.g., energy intake that is less than energy expenditure) and/or weight reduction. Thus, in embodiments, the system uses mobile health (mHealth) technology to target multiple behavior-change interventions for the sleep apnea population and/or individuals in general (e.g., patients who do not necessarily have sleep apnea) to improve health outcomes.

Aspects disclosed herein provide systems for treatment adherence monitoring. The system includes a computing system that includes a processor coupled to non-transitory, computer-readable memory containing instructions executable by the processor to cause the computing system to receive data from a PAP device associated with the use of the device by a wearer; receive sleep-wake tracking data from a wearable device; generate, based on an analysis of the data received from the PAP device and the wearable device data, a score defining adherence to treatment as a percent PAP wear time relative to the time in bed and display the score and data associated with at least one health metric via an interface on a mobile device. Time in bed is used as a denominator in the percent adherence calculation because it is more meaningful to patients (i.e., end users) in order to meet their 100% of PAP use during the entire time spent in bed for sleeping. In some embodiments, time asleep, i.e., total sleep duration or different sleep stages (e.g., rapid eye movement sleep) may be used as the denominator. The latter embodiment is meaningful for providers to track the adherence and efficacy of treatment.

As mentioned, in some embodiments, the data received from the PAP device indicate the percentage of time the PAP mask was worn during the actual time spent asleep during the time spent in bed. Further, in some embodiments, the sleep tracking data represent total sleep time. In some embodiments, the systems disclosed herein include an automated algorithm such that the algorithm determines an overlap between the time the PAP mask is worn and a time the wearer is in bed for sleeping. Further, in some embodiments, the algorithm expresses the adherence to treatment as a percentage based on the time the PAP mask was worn to a total sleep duration. In some embodiments, the sleep tracking data is a time the wearer was in bed for sleeping, a total sleep duration, and a time the wearer was in a sleep stage, all of which can also be expressed separately for daytime naps and night time sleep period. In specific embodiments, the sleep stage is one or more of rapid eye movement (REM) sleep and a non-rapid eye movement (NREM) sleep stages (e.g., NREM Stages 1, 2, or 3).

Notably, the data associated with a health metric include one or more of weight, height, age, gender, race/ethnicity, body mass index (BMI), activity, nutrition, socio-economic status, profession, and/or geographical location, heart rate, prior PAP adherence, measures of the severity of sleep apnea (e.g., apnea-hypopnea index (AHI), oxygen desaturation index (ODI)), average sleep over time, diet, energy intake, exercise, energy expenditure metrics, energy balance, resting metabolic rate, weight change, in some embodiments.

In some embodiments, the score and the health metric are presented as feedback to a wearer. In certain embodiments, the sleep tracking data are produced by a wearable device. In some embodiments, the data associated with a health metric are produced by a wearable device. In some embodiments, the wearable device is a sleep tracker configured to monitor sleep apnea (e.g., by capturing multiple biosignals relevant to sleep apnea). In some embodiments, the wearable device is an actigraphy sensor. In some embodiments, the wearable device is a heart rate sensor and/or a motion sensor.

In some embodiments, the processor further causes the computing system to send a notification to the wearer via a mobile device, wherein the notification is a reminder to use the PAP device. In some embodiments, the processor further causes the computing system to send a notification to the wearer via a mobile device, wherein the notification is a reminder of bedtime or wake-up time schedules or sleep hygiene related personalized tasks. For example, for successful sleep extension intervention, using an AI-assisted algorithm (e.g., using individual habitual sleep patterns) along with the healthcare provider input (as needed) the wearer can receive “Sleep Home Works” (e.g., a few item tasks) to improve their sleep habits. These “Sleep HomeWorks” can be updated/revised (as needed) and tailored to patient's needs based on prior 2-week patterns using AI-e.g., assisted algorithm or provider input or user can change them as well. The systems disclosed herein also provide embodiments in which the computing system is configured to track percent sleep adherence (relative to healthy sleep recommendations) to treatment over time. For example, the recommended time in bed duration can be set at any value between 7 to 9 hours based on patient's age and sleep habits and preferences. For example, older individuals will be at the lower end of the spectrum and younger individuals will be on the higher end. Additionally, in some embodiments, the processor is provided locally on the mobile device or on a server remote.

The computing system may be configured to communicate and exchange data over a network, in some embodiments. Further, the system may include a database to house data produced by the PAP device and the wearable device. In some embodiments, the wearable device is a watch or other sleep/activity tracker, sleep tracking wearable device, and/or sleep apnea sensor worn on other body sites e.g., ring worn on finger.

In other aspects, methods are disclosed for providing treatment adherence monitoring in a subject. The methods may include receiving data from a PAP device associated with use of the device by a user; receiving, from one or more different devices, sleep tracking data; generating, based on an analysis of the data received from the PAP device and the sleep tracking data, a score defining a percentage of adherence to PAP use as a function of time in bed or total sleep time, or sleep stages, and displaying the score on a mobile device.

In some embodiments of the disclosed method, the mobile device is connected, via a cloud-based platform, to a computer server including a processor coupled to non-transitory, computer-readable memory containing instructions executable by the processor to cause the computer server to receive and analyze the data to generate the score. For example, in some embodiments the sleep tracking data are indicative of sleep time. The method may include an automated algorithm, wherein the algorithm determines an overlap between the time the PAP mask is worn and a time the wearer is in bed (including naps) or time asleep.

In specific embodiments of the methods disclosed herein, the data associated with a health metric include one or more of weight, height, age, gender, race/ethnicity, BMI, activity, nutrition, socio-economic status, profession, and/or geographical location, heart rate, prior PAP adherence, measures of the severity of sleep apnea (e.g., apnea-hypopnea index (AHI), oxygen desaturation index (ODI)), average sleep over time, diet, energy intake, exercise, energy expenditure metrics, energy balance, resting metabolic rate, weight change.

The sleep tracking data may be received from a wearable device, such as a watch, or other dedicated modality for measuring sleep data. The processor may further cause the computer server to send a notification to the user via the mobile device, in some embodiments of the method, such that the notification includes a reminder to use the PAP device and/or a reminder of bedtime or wake-up time schedules or sleep hygiene related personalized tasks. For example, for successful sleep extension intervention, using the AI-assisted algorithm (e.g., using individual habitual sleep patterns captured electronically by the app via survey or objectively as data collection progresses) along with the healthcare provider input (as needed) the person can receive “Sleep HomeWorks” (a few item tasks) to improve their sleep habits. These “Sleep HomeWorks” can be updated/revised (as needed) and tailored to patient's needs based on prior 2-week patterns using AI-assisted algorithm or provider input or user can change them as well. In some embodiments, the notification is tailored to habitual bedtimes from the sleep tracker and includes a reminder to use the PAP device and to encourage PAP wear based on individual percent PAP adherence data, thus encouraging and/or improving PAP treatment adherence. In some embodiments, the notification is tailored to habitual bedtimes from the sleep tracker and includes a reminder to bedtime or wake-up time schedules or sleep hygiene related personalized tasks, thus encouraging and/or improving sleep times to achieve sleep optimization. In some embodiments, the bedtime or wake-up time schedules can be modified to extend sleep (e.g., increase sleep duration), thus encouraging and/or improving sleep habits to meet goals that incrementally extend sleep duration over a period of time in order to optimize sleep patterns.

In some embodiments of the methods disclosed herein, the computer server is configured to track a user's percentage of adherence to treatment and to store data in a database.

In another aspect, a method includes: accessing, by one or more processors, first data from a positive airway pressure (PAP) device, the first data indicating one or more first time intervals during which a mask of the PAP device was worn by a subject; accessing, by the one or more processors, second data from a sensor apparatus worn by the subject, the second data indicating one or more second time intervals during which the subject was in bed; determining, by the one or more processors based on the first data and the second data, that the subject was in bed for a first length of time and that the mask was worn by the subject during a second length of time while the subject was in bed; determining, by the one or more processors, a PAP adherence metric based on the first length of time and the second length of time; and causing, by the one or more processors, the PAP adherence metric to be presented to a user.

Implementations of this aspect can include one or more of the following features.

In some implementations, determining the PAP adherence metric can include determining a ratio between the first time and the second time.

In some implementations, the one or more second time intervals can be within a pre-determined range of time. The pre-determined range of time can be selected by at least one of the subject or the user.

In some implementations, the PAP device can be at least one of a continuous positive airway pressure (CPAP) device, a bilevel positive airway pressure (BiPAP) device, or an adaptive servo-ventilation (ASV) device.

In some implementations, the user can be at least one of the subject or a health care provider.

In some implementations, causing the PAP adherence metric to be presented to the user can include generating a graphical user interface using an electronic device. Further, the graphical user interface can indicate the PAP adherence metric.

In some implementations, the method can also include determining whether the PAP adherence metric is less than a threshold value, and selectively presenting a notification to the user based on the determination whether the PAP adherence metric is less than the threshold value.

In some implementations, the method can further include determining that the PAP adherence metric is less than a threshold value, and responsive to determining that the PAP adherence metric is less than the threshold value, presenting a notification to the user.

In some implementations, the method can also include generating, based on the PAP adherence metric, one or more health recommendations for the subject, and causing the one or more health recommendations to be presented to the user.

In some implementations, generating the one or more health recommendations for the subject can include, responsive to determining that the PAP adherence metric is less than the threshold value, generating a first recommendation to the subject to increase a percentage of time that the subject wears the mask while the subject is in bed.

In some implementations, the sensor apparatus can include one or more accelerometers.

In some implementations, the method can further include accessing third data from the sensor apparatus, the second data indicating one or more third time intervals during which the subject was sleeping; determining, based on the third data, that the user was sleeping for a third length of time; and causing the third length of time to be presented to the user.

In some implementations, the method can further include determining a ratio between the third length of time and a target length of time, and causing the ratio between the third length of time and the target length of time to be presented to the user.

In some implementations, the target length of time can be selected based on data representing a sleep pattern of the subject.

In some implementations, the method can include determining whether the third length of time is less than a target length of time, and selectively presenting a notification to the user based on the determination whether the third length of time is less than the target length of time.

In some implementations, the method can further include determining that the third length of time is less than a threshold length of time, and responsive to determining that the third length of time is less than the target length of time, presenting a notification to the user.

In some implementations, the method can further include generating, based on the time length of time, one or more health recommendations for the subject; and causing the one or more health recommendations to be presented to the user.

In some implementations, generating the one or more health recommendations for the subject can include, responsive to determining that the third length of time is less than the threshold length of time, generating a first recommendation to the subject to increase the subject's sleep duration.

In some implementations, generating the one or more health recommendations for the subject can include determining at least one of: a recommended bedtime for the user, or a recommended wake time for the user.

In some implementations, at least one of the recommended bedtime for the user or the recommended wake time for the user can be determined based on data representing a sleep pattern of the subject.

In some implementations, generating the one or more health recommendations for the subject can include generating a first recommendation to the subject to meet a weight loss goal, wherein the first recommendation comprises an indication to increase the subject's sleep duration.

Other implementations are directed to systems, devices and non-transitory, computer-readable media including computer-executable instructions for performing the techniques described herein.

### BRIEF DESCRIPTION OF THE DRAWINGS

FIGS. 1A and 1B illustrate examples of PAP use data from a patient from two different nights.

FIGS. 1C and 1D illustrate examples of PAP use data from a patient over several nights.

FIG. 2A illustrates night-to-night variability in “time in bed” over a period of 21 days in a patient, and the resulting differences between a first adherence metric (e.g., PAP mask wear in hours) and a second, improved percent PAP adherence metric, as disclosed herein.

FIG. 2B illustrates night-to-night variability in “time in bed” over a period of 10 days in another patient, and the resulting differences between a first adherence metric (e.g., PAP mask wear in hours) and a second, improved percent PAP adherence metric, as disclosed herein.

FIG. 2C illustrates night-to-night variability in “time in bed” over a period of 5 to 6 months in four different patients (numbered 2, 8, 17, and 25), showing that time in bed is highly variable within and between individuals.

FIG. 3 shows an example process for determining a PAP (e.g., CPAP BiPAP, and/or ASV) adherence metric.

FIGS. 4A and 4B are block diagrams illustrating embodiments of disclosed systems.

FIGS. 5A and 5B are block diagrams illustrating embodiments of disclosed systems.

FIGS. 6A and 6B illustrate embodiments of a mobile device interface of the disclosure.

FIG. 7 illustrates one embodiment of a mobile device interface of the disclosure.

FIGS. 8A and 8B illustrate example embodiments of methods disclosed herein.

FIGS. 9A and 9B illustrate embodiments of an application interface.

FIG. 10 illustrates an embodiment of an application interface.

### DETAILED DESCRIPTION

By way of overview, the disclosure is directed to systems and methods for treatment adherence monitoring. Systems and methods disclosed herein provide techniques for defining adherence to PAP treatment as a percentage of time spent in bed for sleeping. In some embodiments, adherence to PAP treatment refers to a percentage of total sleep time. Specifically, embodiments of the disclosure provide a metric to determine adherence to therapy as a function of hours spent in bed (e.g., in which time in bed may include either asleep or awake states, such as periods of awakening while in bed). In contrast, at least some existing methods use only absolute hours of PAP wear (e.g., “mask-on time”) without information regarding time in bed. Systems and methods disclosed herein use mobile and wireless technologies in combination with improved measurement techniques to support achieving improved health goals, health outcomes, and healthcare services. Thus, systems and methods disclosed herein may be implemented into clinical practice as a cost-effective means of fostering treatment adherence, better patient care and management individually scalable to a large and diverse patient population.

In addition to PAP treatment, systems and methods disclosed herein provide techniques for defining adherence to other types of treatment (e.g., diet and exercise intervention for weight loss) or other types of health management (e.g., management of overweight/obesity) through sleep optimization (e.g., obtaining a healthy sleep duration and quality). Specifically, embodiments of the disclosure provide a metric to determine adherence to healthy sleep patterns by defining time spent in bed sleeping as a percentage of healthy sleep recommendations. In contrast, at least some existing methods fail to consider the effect of sleep hygiene (healthy sleep duration or quality) on disease, health status, health goals, health outcomes, and healthcare services related to weight loss intervention. Thus, systems and methods disclosed herein may be implemented into clinical practice or self-care to foster adherence to healthy behavior change.

As described above, sleep apnea, such as obstructive sleep apnea (OSA) or central sleep apnea, is a sleep disorder characterized by recurrent complete or partial upper airway obstruction resulting in reduced oxygen levels at night, sleep fragmentation, and poor sleep quality. PAP (e.g., CPAP, BiPAP, and/or ASV) works by delivering continuous air pressure, preventing upper airway closures during sleep. In at least some cases, effective sleep apnea treatment includes all-night PAP use (e.g., during the entire time spent in bed to optimally and completely eliminate respiratory events and improve sleep quality. However, at least some current clinical guidelines categorize patients as adherent to treatment based on a cut-off point of 4 hours of PAP use. This adherence definition is widely accepted but it is primarily based on some expert opinion and remains in common use today despite a lack of evidence showing that it is sufficient or has any health benefits compared to other more specific measures of usage duration. Further, poor adherence to PAP treatment is a common problem, and when PAP adherence is defined by PAP use of more than 4 hours per night, adherence is variable among individuals with a large proportion of patients being non-adherent to the treatment.

Several interventions, such as educational materials, motivational interviewing, remote monitoring, and other mHealth technologies have been used to promote PAP adherence but have provided limited clinical translation to routine patient care. Additionally, weight loss is often recommended for sleep apnea patients but remains a major challenge in this patient population. To date, the effectiveness of eHealth interventions in improving PAP adherence remains uncertain, highlighting the need for newly designed technology-supported interventions in sleep apnea. Thus, systems and methods disclosed herein address the critical need for tools to address these important clinical barriers in sleep apnea management.

For example, in at least some implementations, the goal for effective sleep apnea treatment is all-night PAP use (e.g., 100% of the time spent in bed) to optimally treat respiratory events, hypoxia, and sleep fragmentation, and thus prevent adverse health effects that are associated with hours slept without wearing PAP. Moreover, in clinical practice, according to Medicare criteria, patients who wear their PAP for 4 hours or more per night for 70% of the nights are considered “adherent” to therapy. However, as described above, for complete efficacy, the goal for PAP usage is wearing the mask 100% of the time while in bed sleeping.

In at least some existing PAP adherence tracking systems (e.g., smartphone apps), a system relies only on mask wear time to determine PAP treatment adherence, which is often referred to as therapy hours. These existing technologies simply capture the number of hours the PAP mask is worn per night but, unlike the present disclosure, do not account for hours spent in bed without wearing the PAP device, and thus therapy (or lack thereof) is not accurately captured.

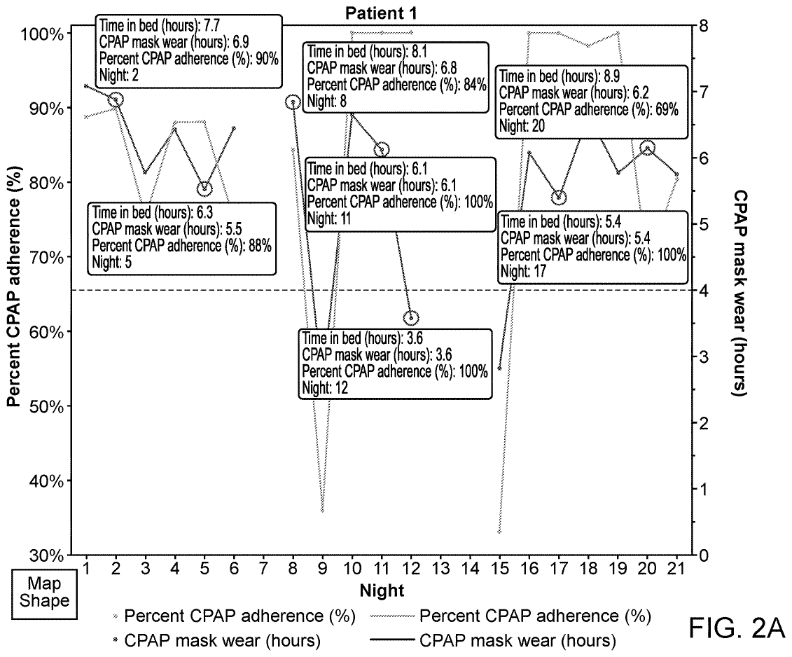

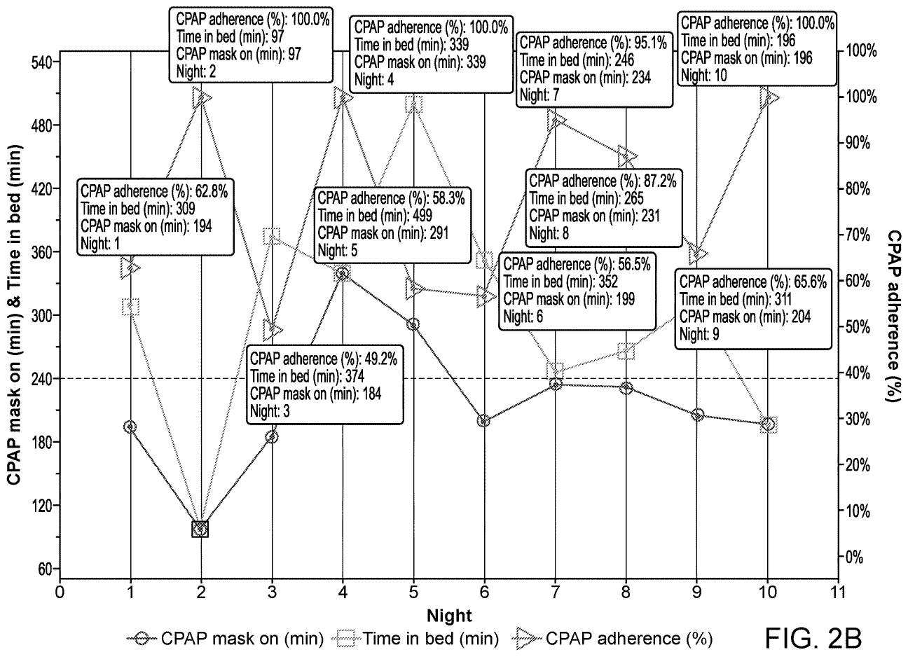

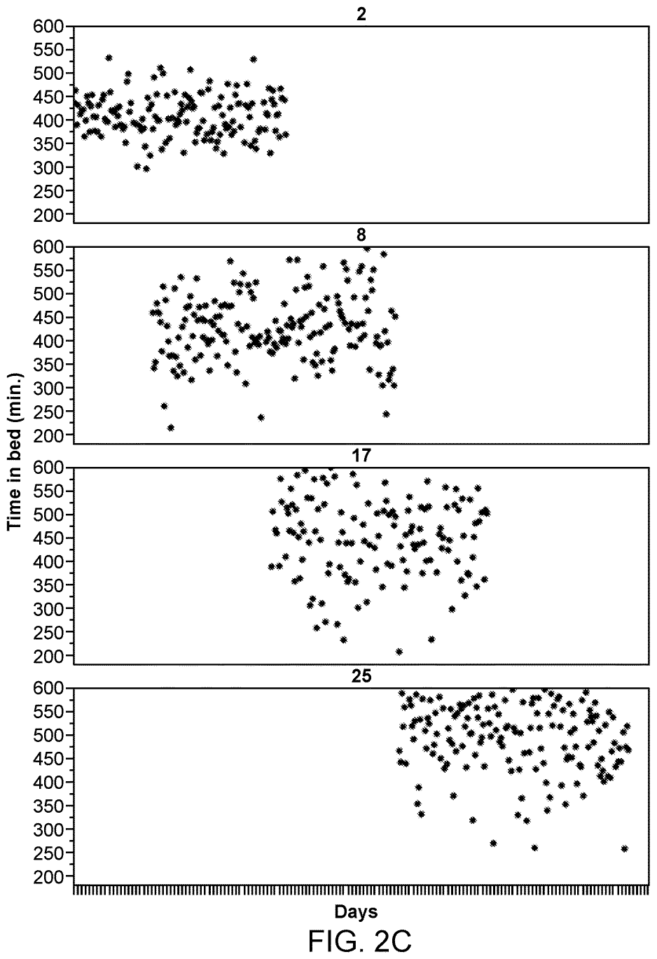

The policy recommendations with adherence defined only as the PAP mask use also have implications for health equity given the known racial/ethnic and socioeconomic differences in sleep patterns, particularly sleep duration. The use of the 4-hour PAP wear as a cut-off point defining adequate treatment adherence is arbitrary and could be misleading because the sleep patterns can vary considerably from night to night and between individuals (see, e.g., FIG. 2C, as described below).

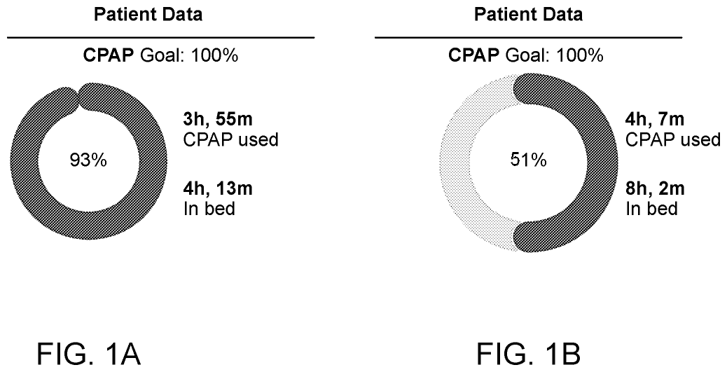

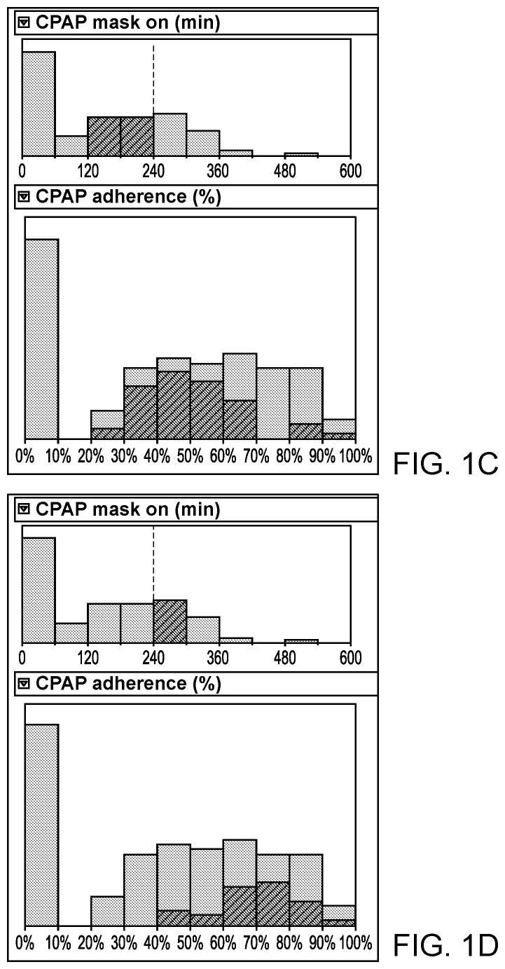

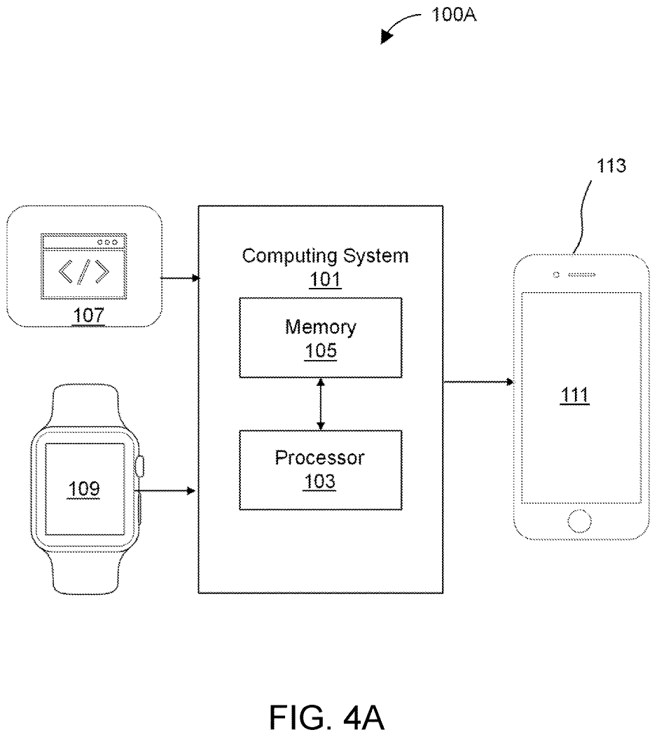

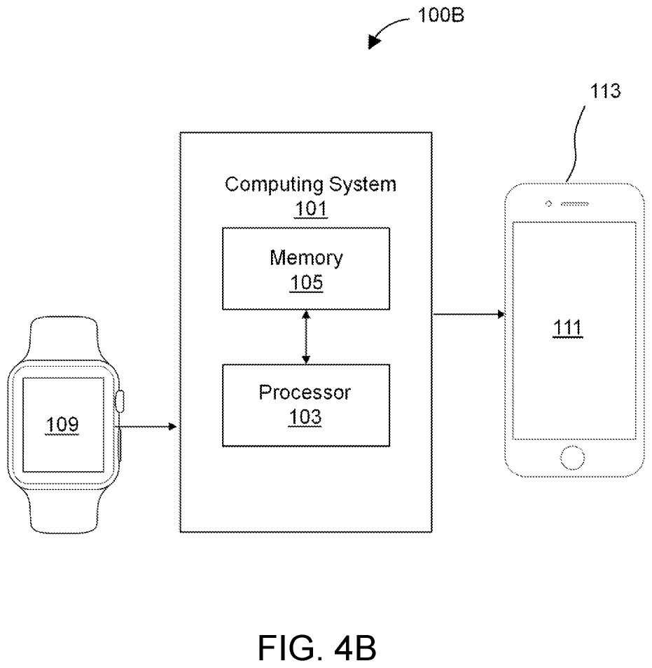

FIGS. 1A and 1B illustrate an example of data from two different nights in a patient. As illustrated, PAP mask wear times are quite similar on both nights, yet percent adherence the PAP is highly different. For example, the mask was worn (e.g., PAP use) for 3 h 55 min (FIG. 1A) and 4 h 7 min (FIG. 1B). Based on the current clinical threshold that defines 4-hours of PAP wear as adherent, the patient illustrated in FIG. 1A would be considered PAP “non-adherent” on the night with 3 h 55 min of PAP wear. The patient illustrated in FIG. 1B would be considered PAP “adherent” on the night with 4 h 7 min of PAP wear.

Methods and systems disclosed herein provide for a more accurate PAP adherence metric by taking into account the time patient spent in bed. Time in bed captures a block of time spent for the purpose of sleeping, and may include both asleep and awake time. For example, naps, including naps occurring in a chair or couch (e.g., out of bed), may be categorized as sessions for the purposes of determining “time in bed” or rest time for sleeping. In some embodiments, systems and methods disclosed herein take into account total sleep duration (e.g., the captured actual sleep during time in bed).

As disclosed herein, when time in bed is considered using the percent PAP tracking systems and methods of the disclosure, the patient in FIG. 1A has a true PAP adherence relative to the time in bed of 93% on the night shown. This patient would thus be considered “adherent”. Notably, the patient illustrated in FIG. 1B would have true PAP adherence relative to time in bed of 51% for the night shown. Yet, both patients have very similar time in bed of 3 h 55 m and 4 h 7 min, respectively. Thus, the PAP adherence metrics of systems and methods disclosed herein, which take into account the time in bed, provide important, clinically meaningful information about PAP adherence, which is not captured by current art PAP adherence tracking systems and provides clinically meaningful, actionable feedback to improve treatment and health outcomes

In another example, based on the current 4-hour PAP adherence threshold, a patient who wears a PAP mask for 5 hours but spends 8 hours in bed per night would be considered adherent. Using these absolute hours of mask wear, as does the prior art, the treatment for this patient is improperly classified. Using the systems and methods disclosed herein, this patient's true PAP adherence should be only 63% as a percentage of time spent in bed. Hence, the patient's treatment adherence would be considered suboptimal, and thus the patient should be advised clinically to increase their PAP usage.

As further examples, FIGS. 1C and 1D show histograms (e.g., distribution of nights) for a patient representing (i) PAP (e.g., CPAP) mask on time and (ii) PAP (e.g., CPAP) percent adherence data collected over several months. As shown in FIG. 1C, the patient wore the mask between 120 and 240 minutes for several nights (indicated by the hashed bars in the top histogram). Based on the current clinical threshold that defines 4-hours of PAP wear as adherent, the patient would be considered non-adherent during each of those nights. However, the patient's PAP adherence varied greatly during those nights (e.g., between 20% and 100%) (as indicated by the hashed bars in the bottom histogram), when defined as a percentage of time in bed for sleeping (e.g., percent adherence metric) due to variations in the length of time that the user was in bed.

Further, as shown in FIG. 1D, the same patient wore the mask between 240 and 300 minutes for several nights (indicated by the hashed bar in the top histogram). Based on the current clinical threshold that defines 4-hours of PAP wear as adherent, the patient would be considered adherent during each of those nights. However, the patient's PAP adherence varied greatly during those nights (e.g., between 40% and 100%) (as indicated by the hashed bars in the bottom histogram) when defined as a percentage of time in bed for sleeping (e.g., percent adherence metric) due to variations in the length of time that the user was in bed.

These examples illustrate that current adherence tracking based on PAP mask-on time does not always provide an accurate information on patient's true adherence to therapy and, in some cases, may be misleading for the patient or the health care provider in guiding sleep apnea management and treatment or deciding on further action or patient counseling to improve outcomes.

FIG. 2A illustrates the night-to-night variability in “time in bed” over a period of 21 days for a patient. Systems disclosed herein recognize that sleep patterns are highly individual, and can change from night to night even in the same person. Specifically, FIG. 2A shows the PAP mask wear and percent PAP adherence metric (e.g., CPAP mask wear and percent CPAP adherence metric) in a single patient monitored over 21 consecutive nights. As illustrated, the adherence metric based on the absolute hours of mask wear (circled points for examples), as is the case for the current metric, does not accurately capture the true PAP adherence of the patient. The percent adherence metric disclosed herein, which in this case takes into account the time in bed, presents a more accurate measure of adherence to PAP treatment by the patient.

For example, on night 12, the patient wears PAP mask for 3.6 hours, and time in bed is also 3.6 hours so adherence to PAP treatment is 100%, yet this patient is considered as “non-adherent” according to current Medicare adherence definitions. Likely this patient's PAP machine would be discontinued by insurance companies if this pattern is sustained during the patient's initial 30-day trial period. FIG. 2A illustrates that data comparing the percent adherence metric of systems disclosed herein to the absolute hours metric used by the prior art show that the percent PAP adherence metric of the disclosure provides more accurate information, and previously unavailable insights, that are different from the prior art.

FIG. 2B illustrates the night-to-night variability in “time in bed” over a period of 10 days for another patient. As illustrated, the adherence metric based on the absolute hours of mask wear (red circle points for examples), as is the case for the current metric, does not accurately capture the true PAP adherence of the patient. The percent adherence metric disclosed herein (green triangle points for examples), which in this case takes into account the time in bed (blue square points for examples), presents a more accurate measure of adherence to PAP treatment by the patient.

Therefore, systems and methods disclosed herein recognize that an accurate calculation of adherence to PAP use includes determining a “denominator” (e.g., hours spent in bed) that differs from simple duration of “mask-on time” as the measure for adherence. Thus, the disclosure distinguishes measuring device use or adherence (e.g., therapy use as defined by current protocols) from percent of time the device is worn in relation to the total time spent in bed for sleeping. In at least some implementation, time in bed can refer to a block of time in bed for sleeping. In some embodiments, the denominator used is the total sleep time or sleep stage.

Additionally, in some embodiments, systems and methods disclosed herein use at least one health metric in calculating the score defining adherence to treatment as a percentage of total sleep time. The embodiments of systems and methods disclosed herein personalize the adherence to treatment score based on one or more individual health metrics that may affect sleep patterns. As such, individual sleep patterns are an important aspect of the denominator calculation used in the disclosure.

Thus, systems and methods disclosed herein provide for implementing more accurate PAP adherence goals, patient monitoring, and patient management guidelines by quantifying PAP wear time in proportion to the time spent in bed. The percent PAP adherence tracking is defined as percent PAP wear time relative to objectively assessed time in bed.

Specifically, systems and methods disclosed herein utilize wearable mHealth devices for at-home monitoring of sleep via accelerometry-based technology in combination with PAP use data to provide a more accurate assessment of treatment adherence. Combining such mHealth technology with PAP use data significantly improves sleep apnea treatment adherence guidelines and fulfills a critically unmet need for patients and healthcare providers for disease treatment/management and prevention and/or reversal of associated health risks.

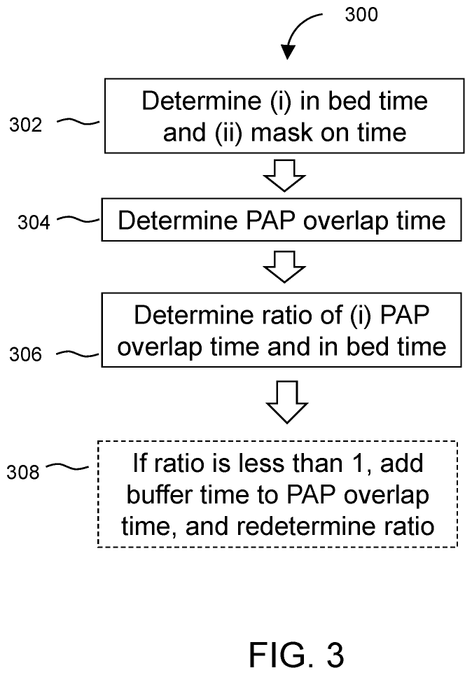

FIG. 3 shows an example process 300 for determining a PAP (e.g., CPAP, BiPAP, and/or ASV) adherence metric. According to the process 300, a system determines (i) the amount of time that the patient is in bed and (ii) the amount of time that the patient is wearing the mask of the PAP device (302).

Further, the system determines a PAP overlap time (304). The PAP overlap time refers to the amount of time that the patient was wearing the mask of the PAP device while they were in bed (e.g., the overlap time between the amount of time that the patient is in bed and (ii) the amount of time that the patient is wearing the mask).

Further, the system determines the ratio of (i) the PAP overlap time and (ii) the amount of time that the patient was in bed (306). In some implementations, the ratio can be capped by 100% or 1. This ratio represents the PAP adherence metric. In some implementations, the PAP adherence metric can be expressed as this ratio. In some implementations, the PAP adherence metric can be expressed as a percentage value.

In some implementations, if the ratio is less than 1, the system can add a pre-determined amount of buffer time to PAP overlap time (e.g., 15 minutes or less) and recalculate the ratio (308). This can be beneficial, for example, in not discouraging patients if they are nearly (but not completely) adherent in wearing the mask while they are in bed. This can also be beneficial, for example, to account for potential inaccuracies in tracking in-bed time and/or mask on time. In some implementations, the system can refrain from adding a buffer time to the PAP overlap time (e.g., such that only the originally determined PAP adherence metric is reported).

FIG. 4A is a block diagram illustrating at least one embodiment of systems disclosed herein. Aspects of the disclosure include a system for treatment adherence monitoring. For example, the system 100A may include a computing system 101 having a processor 103 coupled to non-transitory, computer-readable memory 105 containing instructions executable by the processor 103. The instructions may cause the computing system 101 to receive data from a PAP device 107 (e.g., a CPAP device, a BiPAP device, and/or an ASV device) associated with use of the device by a wearer. The computing system also receives sleep tracking data from a wearable device 109. The instructions cause the computing system to generate, based on the analysis of the data received from the PAP device 107 and the sleep tracking data received from the sleep wearable device 109, a score defining adherence to treatment as a percentage of time in bed. The computing system may then display the score and data associated with at least one health metric via an interface 111 on a mobile device 113.

It should be noted that embodiments of the system 100A of the present disclosure include computer systems, computer operated methods, computer products, systems including computer-readable memory, systems including a processor and a tangible, non-transitory memory configured to communicate with the processor, the tangible, non-transitory memory having stored instructions that, in response to execution by the processor, cause the system to perform steps in accordance with the disclosed principles, systems including non-transitory computer-readable storage medium configured to store instructions that when executed cause a processor to follow a process in accordance with the disclosed principles.

In some embodiments, the processor may be provided locally on the mobile device or on a remote server. The mobile device 113 may generally include a computing system. As shown, the computing system 101 includes one or more processors, such as processor 103. The processor 103 may be embodied as any type of processor capable of performing the functions described herein. For example, the processor may be embodied as a single or multi-core processor(s), digital signal processor, microcontroller, or other processor or processing/controlling circuit.

In some embodiments, the mobile device 113 may maintain one or more application programs, databases, media and/or other information in a main and/or secondary memory. The memory may include, for example, a hard disk drive and/or removable storage drive, representing a floppy disk drive, a magnetic tape drive, an optical disk drive, etc. A removable storage drive may read from and/or write to a removable storage unit in any known manner. The removable storage unit may represent a floppy disk, magnetic tape, optical disk, etc. which is read by and written to by a removable storage drive. As will be appreciated, a removable storage unit may include a computer usable storage medium having stored therein computer software and/or data.

The computing system 101 further includes main memory 105, such as random access memory (RAM), and may also include secondary memory. The main memory 105 may be embodied as any type of device or devices configured for short-term or long-term storage of data such as, for example, memory devices and circuits, memory cards, hard disk drives, solid-state drives, or other data storage devices. Similarly, the memory 105 may be embodied as any type of volatile or non-volatile memory or data storage capable of performing the functions described herein. The computing system 101 may further include one or more application programs directly stored thereon. The application program(s) may include any number of different software application programs, each configured to execute a specific task.

FIG. 4B is a block diagram illustrating at least one embodiment of systems disclosed herein. Aspects of the disclosure include a system for treatment adherence monitoring. For example, the system 100B may include a computing system 101 having a processor 103 coupled to non-transitory, computer-readable memory 105 containing instructions executable by the processor 103. The computing system receives sleep tracking data from a wearable device 109. The instructions cause the computing system to generate, based on the analysis of the sleep tracking data received from the sleep wearable device 109, a score defining adherence to treatment as a percentage of time in bed. The computing system may then display the score and data associated with at least one health metric via an interface 111 on a mobile device 113.

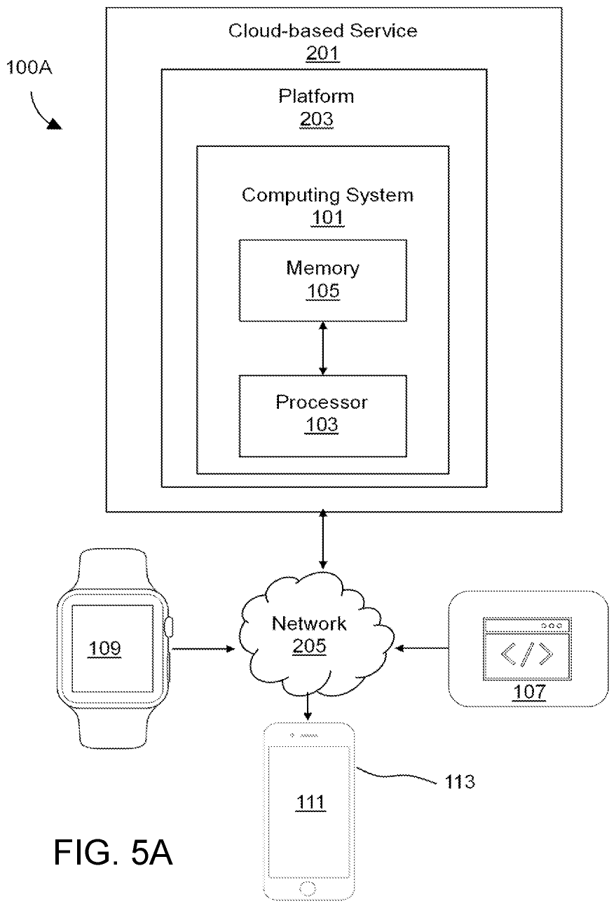

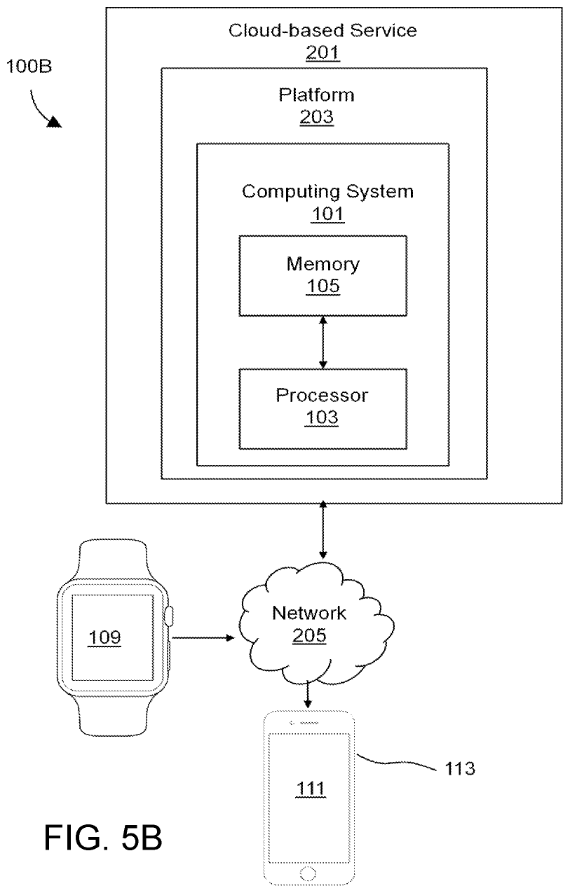

FIG. 5A is a block diagram illustrating one embodiment of systems disclosed herein. In some embodiments, the system 100A includes a platform 203 embodied on an internet-based computing system 101. For example, the platform 203 may be embodied on a cloud-based service 201. The platform 203 is configured to receive data, specifically PAP data, sleep-tracking data, and health-related metric data, analyze the received data, and generate a score defining an adherence to treatment. The score is communicated over a network 205 to an interface 111 displayed on a mobile device 113. Thus, in some embodiments, the computing system is configured to communicate and exchange data over a network.

The network 205 may represent, for example, a private or non-private local area network (LAN), personal area network (PAN), storage area network (SAN), backbone network, global area network (GAN), wide area network (WAN), or collection of any such computer networks such as an intranet, extranet or the Internet (e.g., a global system of interconnected network upon which various applications or service run including, for example, the World Wide Web).

The network 205 may be any network that carries data. Non-limiting examples of suitable networks that may be used as network 205 include Wi-Fi wireless data communication technology, the internet, private networks, virtual private networks (VPN), public switch telephone networks (PSTN), integrated services digital networks (ISDN), digital subscriber link networks (DSL), various second generation (2G), third generation (3G), fourth generation (4G) cellular-based data communication technologies, Bluetooth radio, Near Field Communication (NFC), the most recently published versions of IEEE 802.11 transmission protocol standards as of October 2018, other networks capable of carrying data, and combinations thereof. In some embodiments, the network 205 is chosen from the internet, at least one wireless network, at least one cellular telephone network, and combinations thereof. As such, the network 205 may include any number of additional devices, such as additional computers, routers, and switches, to facilitate communications. In some embodiments, the network 205 may be or may include a single network, and in other embodiments the network 205 may be or include a collection of networks.

The platform 201 may be configured to communicate and share data with a mobile device 113 embodied as any type of device for communicating with the platform 203 and cloud-based service 201, and/or other user devices, such as a wearable device 109, over the network 205. For example, at least one of the user devices may be embodied as, without limitation, a sleep tracker, a scale, a medical device such as a blood pressure cuff, a computer, a desktop computer, a personal computer (PC), a tablet computer, a laptop computer, a notebook computer, a mobile computing device, a smart phone, a cellular telephone, a handset, a messaging device, a work station, a distributed computing system, a multiprocessor system, a processor-based system, and/or any other computing device configured to store and access data, and/or to execute software and related applications consistent with the present disclosure. In the embodiments described here, the mobile device 113 is generally embodied as a smartphone or tablet. However, it should be noted that one or more devices 113 may include a computer, a desktop computer, a personal computer (PC), a tablet computer, a laptop computer, a notebook computer, and the like.

FIG. 5B is a block diagram illustrating one embodiment of systems disclosed herein. In some embodiments, the system 100B includes a platform 203 embodied on an internet-based computing system 101. For example, the platform 203 may be embodied on a cloud-based service 201. The platform 203 is configured to receive data, specifically sleep-tracking data and health-related metric data, analyze the received data, and generate a score defining an adherence to treatment. The score is communicated over a network 205 to an interface 111 displayed on a mobile device 113. Thus, in some embodiments, the computing system is configured to communicate and exchange data over a network.

In some embodiments, the systems disclosed herein integrate monitoring treatment adherence and monitoring other health metrics using data obtained from the PAP device 107 as well as data obtained from a wearable device 109, such as a sleep tracker. Thus, systems disclosed herein have the ability to combine information from multiple devices. Systems disclosed herein may use the application programming interface (API) for each device to combine data and information from multiple devices. An API is a software intermediary that allows two application to communicate with each other. For example, the devices may be activity trackers and weight scales, for example an activity tracker, and/or a wireless weight scale (using API). Systems disclosed herein may interface with any PAP device 107, for example any device that includes the sensors, communication channels, processors, and actuators required for generating and transferring PAP usage data. In preferred embodiments, the sleep trackers uses 3-axis accelerosensor (e.g., one or more accelerometers), heart rate sensors, respiratory sensors, oxygen sensors, and/or any other physiological sensors to more reliably determine sleep-wake patterns and time in bed, total sleep duration and sleep stages. For example, the sleep stages may be one or more of a stage of non-rapid eye movement (NREM) or quiet sleep such as NREM Stage 1, NREM Stage 2, NREM Stage 3 (deep sleep). The sleep stage may be rapid-eye movement sleep or active (REM) sleep.

In some embodiments, systems disclosed herein include integrated diet, nutrition, activity, and weight-tracking data received and input as health metric data. This data may also be integrated with a companion interventionist web-based dashboard for example a diet and nutrition tracking dashboard. In some embodiments, the system supports behavioral interventions such as weight-loss or activity or sleep extension or PAP adherence coaching. The system may include an interventionist and/or coach-facing module to display features related to monitored health metrics including percent PAP adherence. For example, the interventionist and/or coach-facing module may include information related to research studies or health care team to which the patient or participant belongs and the behaviors targeted for change. The participant-facing or patient-facing interface may include features for behavior change interventions, for example to foster weight loss, healthier diet quality, and physical activity, as well as sleep intervention (e.g., sleep extension) or PAP adherence coaching for encouraging healthier sleep behaviors. In this way, the system may present interventionists and participants both with relevant information from the PAP adherence and sleep tracking platform integrated with diet, activity, and weight tracking features used to tailor health coaching.

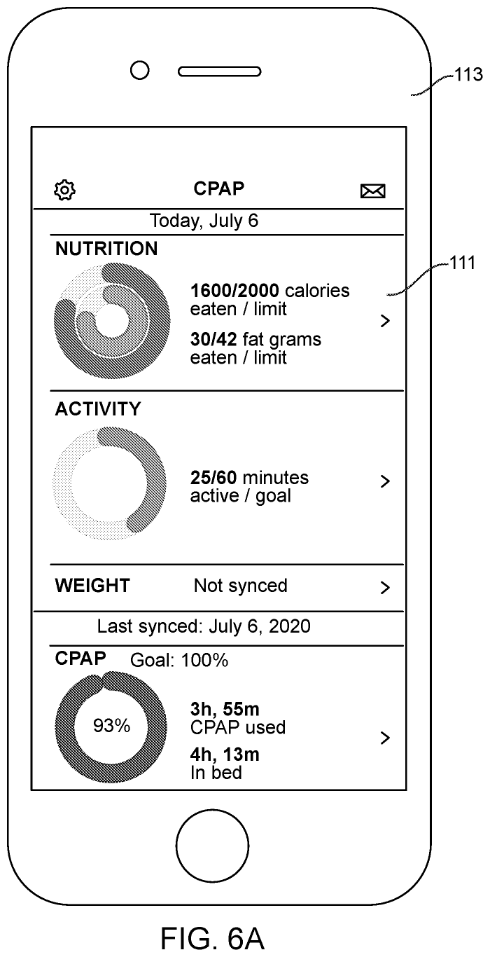

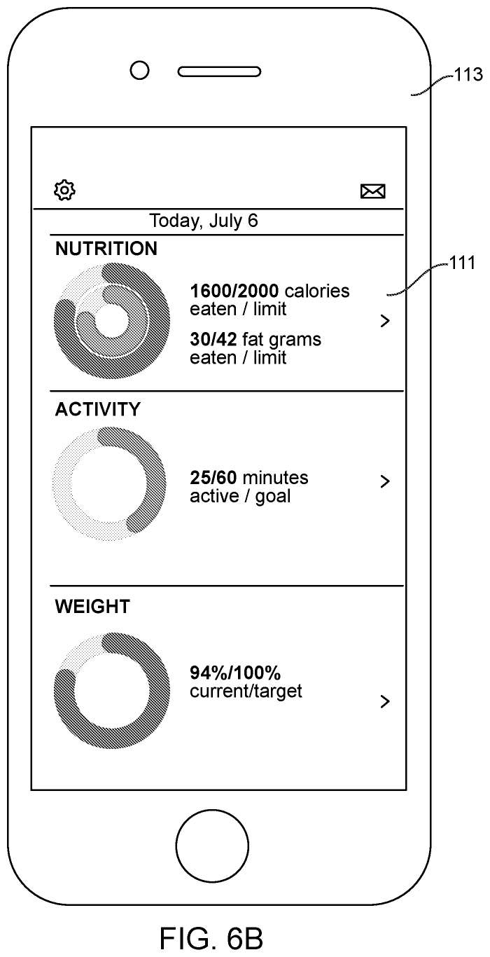

FIG. 6A illustrates one embodiment of a mobile device 113 interface 111 of systems and methods disclosed herein. The system, which may be described as an app, allows a user to track PAP adherence. FIG. 6B illustrates one embodiment of a mobile device 113 interface 111 of systems and methods disclosed herein. In embodiments, the interface may be a user interface not limited to a graphical user interface (GUI) with which a user may interact with the mobile device, such as accessing and interacting with the data displayed by the system. The interface may be a communications interface capable of enabling communications between the mobile device external devices, the computing system, a cloud-based service, and/or the platform. As a communications interface, the interface may be configured to use any one or more communication technology and associated to effect such communication. For example, the communications interface may be configured to communicate and exchange data with the platform, and/or the one or more devices via a wireless transmission protocol including, but not limited to, Bluetooth communication, infrared communication, near field communication (NFC), radio-frequency identification (RFID) communication, cellular network communication, the most recently published versions of IEEE 802.11 transmission protocol standards as of October 2018, and a combination thereof.

In some embodiments, the data received by the computing system from the PAP device is received via the PAP API via Bluetooth communication. The data may indicate the time the PAP mask was worn during the time in bed. Further, the sleep tracking data may represent time in bed (which may or may not include daytime naps), total sleep time and sleep stages (e.g., deep sleep, rapid eye movement sleep). For example, the sleep stages may be one or more of a stage of non-rapid eye movement (NREM) such as NREM Stage 1, NREM Stage 2, or NREM Stage 3 (deep sleep). The sleep stage may be rapid-eye movement sleep or active (REM 4) sleep. The sleep tracking data may be received via the sleep tracking device API via Bluetooth communication.

In addition, in some embodiments, the system allows a user to track data associated with one or more health metrics. For example, the health metric may be any metric that affects to sleep patterns such that calculation of the adherence to treatment may be affected by the sleep pattern. For example, the health metric may be one or more of one or more of weight, height, age, gender, race/ethnicity, BMI, activity, nutrition, socio-economic status, profession, and/or geographical location, heart rate, prior PAP adherence, measures of the severity of sleep apnea (e.g., apnea-hypopnea index (AHI), oxygen desaturation index (ODI)), average sleep over time, diet, energy intake, exercise, energy expenditure metrics, energy balance, resting metabolic rate, weight change, in some embodiments. In this way, the system includes one or more individualized metrics for calculating the adherence to treatment score.

The health metric may be manually received by the computing system or it may be received through an API of a device. For example, a user's AHI may be determined through a sleep study and then entered into the system for use in determining an adherence to treatment when the user is asleep without using the PAP device.

In some embodiments, the system allows a user to also track, nutrition, physical activity, and weight. The interface may display any number of health metrics tracked by the system. In some non-limiting embodiments, the system displays the percent PAP adherence each night. This may be by darkening a portion of a circle's circumference to represent the percentage of time the PAP mask was worn during the time spent in bed or time asleep. For example, the time the PAP mask was worn may be determined and conveyed by the PAP device via, for example, a device API. For example, if a particular PAP device is used, the data may be received via a that particular PAP machine's API. The time the mask was worn may be determined by the device, for example using pressure-flow sensors, and mask-on time. In embodiments, the sleep tracking data are produced by a wearable device. Thus, the time spent in bed may be conveyed by a wearable sleep-activity tracking device, for example by a wrist activity tracker or a watch.

In some embodiments, an automated algorithm analyzes time in bed and total sleep time. The algorithm may first determine the overlap between PAP wear and time in bed using mathematical equations to then express the PAP adherence as percent of time spent in bed (either including both daytime naps and nighttime sleeping, or solely nighttime sleeping) or as a percent time asleep determined from total sleep duration while in bed. In specific embodiments, the automated algorithm determines an overlap between the time the PAP mask is worn and a time the wearer is in bed for sleeping, including naps. Thus, in embodiments, the algorithm checks the blocks of time spent in bed for a wearer and determines the overlap with PAP wear. The algorithm may also be applied to different sleep stages (e.g., deep sleep and rapid eye movement sleep) captured by a wearable device and combined with PAP mask wear data during sleep stages. For example, the sleep stages may be one or more of a stage of non-rapid eye movement (NREM) such as NREM Stage 1, NREM Stage 2, or NREM Stage 3 (deep sleep). The sleep stage may be rapid-eye movement sleep (REM) sleep.

In some embodiments, the computing system is configured to track adherence to treatment over time.

“Goal 100%” may be displayed above on the interface, for example above the circle, to remind participants to wear their PAP the entire time spent in bed. The section may also display in hours and minutes the duration of PAP wear time and time spent in bed separately on any side of the circle. Thus, the PAP tracking in the system provides users with meaningful, actionable feedback that aims to encourage and improve their PAP adherence.

In some embodiments, if a user's mask has a high leak at any point, the system will mark the night with the label “LEAK” and a red dot to alert the participant to troubleshoot mask issues and contact their healthcare provider, as needed.

In some embodiments, the system (e.g., the processor) may further cause the computing system to send a notification to the PAP wearer via a mobile device such that the notification is a reminder to use the PAP device. In some embodiments, the notification is tailored to habitual bedtimes from the sleep tracker and includes a reminder to use the PAP device and to encourage PAP wear based on individual percent PAP adherence data, thus encouraging and/or improving PAP treatment adherence. For example, the system may be configured to send individualized push notifications approximately one hour before the user's bedtime to remind a user to wear the PAP device each night and to sync their sleep-tracker device each morning upon waking. Notifications may be tailored to the user's self-reported bedtime for the first therapy week and thereafter adjusted to their objectively measured average bedtime provided by the sleep-tracker as data become available.

In some embodiments, the system may also send the user encouraging or engaging push notifications tailored to the wearer's specific percent PAP adherence from each night. The frequency of the push notifications may be customized by the wearer and/or the provider. Thus, because the systems and methods disclosed herein provide a more accurate assessment of true adherence, the associated push notifications provide a more meaningful message and feedback to users as compared to prior art systems that simply capture the number of hours the PAP mask is worn.

As noted above, in embodiments, systems disclosed herein integrate nutrition and weight loss goals with the PAP data. For example, the system may include one or more databases. In some embodiments, the system includes a database to house data produced by the PAP device and the wearable device. The system may further include the same or different database or databases for storing data related to diet and exercise, and for tracking diet and exercise data. Nutrition intake may be tracked when a user searches for and selects foods in a database associated with the system or may add custom foods or recipes by recording calorie and fat gram content. Calories and fat gram intake may be totaled each day. Dietary data can also be captured from bar codes, screenshots from the app in some embodiments. Daily calorie and fat gram goals within the nutrition section may be displayed, which are calculated based on the user's weight and/or other health metrics.

Physical activity may be tracked in the system by automatically transferring data from a user's activity tracker, for example a wearable device such as wrist-worn activity tracker, using Bluetooth transfer of data. Physical activities may also be manually entered by searching in the system database, which may include a compendium of physical activities rated by their intensity. Activity may be tracked by selecting the specific activity and its duration. Weight may be tracked automatically by syncing with a smart scale via Bluetooth transfer, for example by using a wireless digital weight scale. Weight may also be manually entered into the app if needed. For every health metric tracked and/or monitored by the system, progress over time may be viewed as a daily, weekly, and/or monthly line graph.

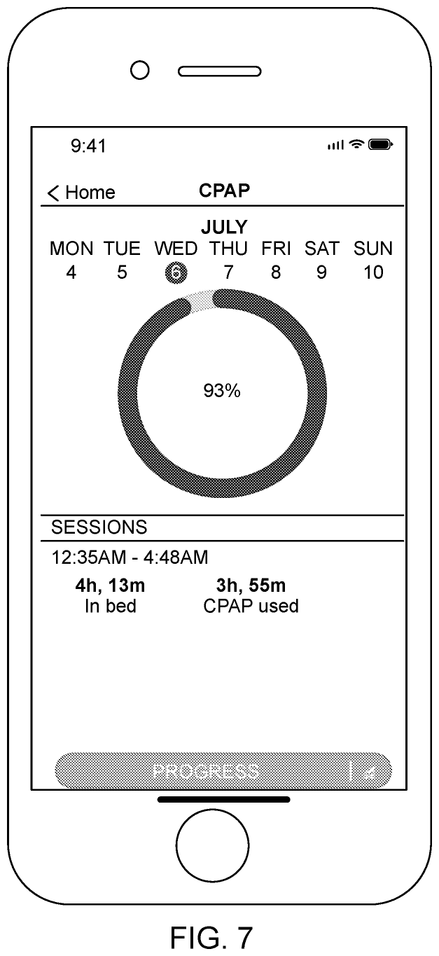

FIG. 7 illustrates an embodiment of the mobile device interface display of systems and methods disclosed herein. As illustrated, the display shows the calculated score defining the user's adherence to treatment for the specific night. The display illustrates the total time in bed for sleeping, as well as the time with the PAP mask on. As illustrated, the user received a calculated adherence to treatment score of 93% indicating the user was close to the goal of 100%. Thus, the PAP adherence metric calculation that accounts for time in bed, provides important, clinically meaningful personalized information about PAP adherence which leads to a more accurate representation of PAP usage for the user.

As described herein, systems and methods of the disclosure recognize that sleep patterns are highly individual and can change from night to night even in the same person. Embodiments of the disclosed systems and methods may use at least one health metric in calculating the score defining adherence to treatment as a percentage of time in bed. In other embodiments, the disclosed systems and methods use at least one health metric in calculation the score defining adherence to treatment as a percentage of total sleep time. The disclosed systems personalize the adherence to treatment score based on one or more individual health metrics that may affect sleep patterns. Thus, individual sleep patterns are an important aspect of the denominator calculation used in the metric for determining treatment adherence. For example the disclosed systems may include sleep staging, e.g. REM sleep, as a metric in the calculation.

In some embodiments, the system includes the ability to characterize the effectiveness of PAP treatment based on residual respiratory events while on treatment. As noted above, the effectiveness of PAP treatment in current protocols (prior art) is determined only during PAP mask-on time. Systems and methods of the disclosure provide for entering the baseline severity of disease into the system at PAP machine set up by a healthcare provider. The baseline severity of disease may be obtained from the diagnostic sleep study (in-lab or home-based) that is required prior to initiation of any PAP treatment.

For example, the disclosed systems recognize that a person who takes their PAP mask off will revert back to a baseline AHI during the corresponding time spent in bed. Thus, in embodiments, systems disclosed herein include an algorithm for calculating a “mask-off AHI”. For example, in embodiments, the algorithm uses the baseline severity of disease (e.g., baseline AHI, baseline ODI) to calculate an estimated AHI (or ODI or other sleep apnea severity metric) based on the baseline assumption. The system then displays the calculated AHI value as part of the adherence to treatment calculation. The display may include a scale upon which the person's calculated AHI falls in relation to clinical levels of severity. For example, the mask on and mask off AHI (or other sleep apnea severity metric) can be shown in color zones from green to red with increasing severity of disease.

Additionally, in some embodiments, the systems disclosed herein may include a real-time wearable sleep apnea tracker monitor that can be used while wearing the PAP device to give a real-time AHI, which in turn may be calculated separately for both mask-on time and mask-off time. For example, the real-time apnea tracker may be a wearable patch applied, for example on the neck of a user, (suprasternal notch or elsewhere on the neck, to detect airflow and blood oxygen saturation and sleep-wake activity (3-axis accelerometer) and heart rate. The sleep apnea monitoring device may be connected to the PAP device or may be a separate device. For example, the device may be a nanosensor worn on the user's neck or other body sites. Communication of the device with the system may be through Bluetooth communication via a device API. In this way, the system provides a calculation of PAP treatment adherence that also gives a real-time AHI calculated for both mask-on time and mask-off time. Thus, the provider can better track the effectiveness of therapy, and monitor the true residual disease severity of a patient who is prescribed PAP therapy. This can subsequently determine patient counseling (e.g., by recommending personalized treatment based on true residual disease) and disease management. Similarly, the patients can monitor their disease severity when the mask is off and be encouraged not to remove the mask to reduce their mask-off AHI and thus be more adherent to therapy to reach their 100% PAP adherence goal.

As noted above, in some embodiments, the system combines the percent PAP adherence score with an integrated weight loss application.

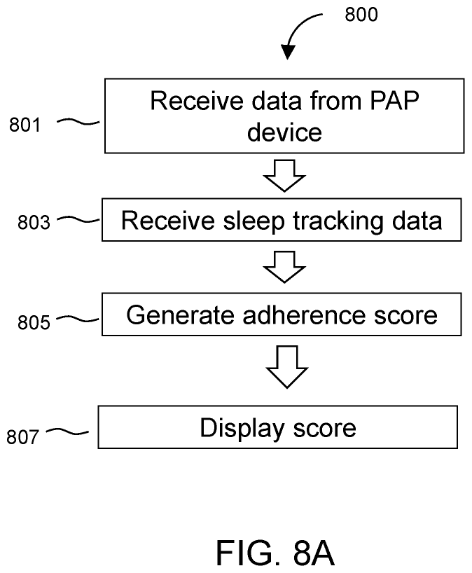

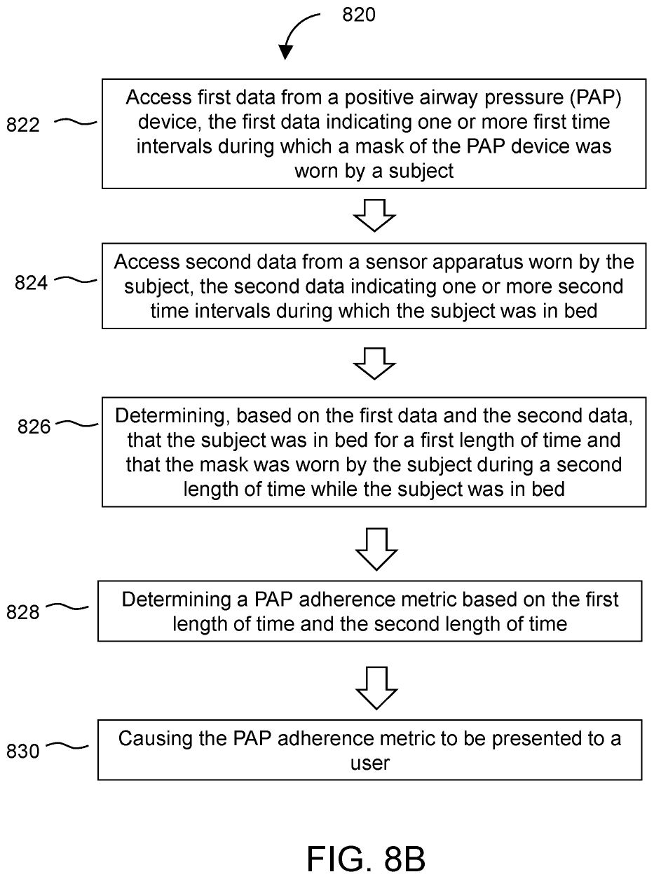

FIG. 8A illustrates one embodiment of a method 800 for providing treatment adherence monitoring disclosed herein. Aspects disclosed include methods for providing treatment adherence monitoring in a subject. For example, the methods may include the steps of receiving data from a PAP device associated during use (801); receiving, from one or more different devices, sleep tracking data (803); generating, based on an analysis of the data received from the PAP device and the sleep tracking data, a score defining a percentage of an adherence to PAP use as a function of sleep time (805); and displaying the score on a mobile device (807).

FIG. 8B illustrates another embodiment of a method 820 for providing treatment adherence monitoring disclosed herein.

In the method 820, a system accesses first data from a positive airway pressure (PAP) device, the first data indicating one or more first time intervals during which a mask of the PAP device was worn by a subject (822).

Further, the system accesses second data from a sensor apparatus worn by the subject, the second data indicating one or more second time intervals during which the subject was in bed (824).

Further, the system determines, based on the first data and the second data, that the subject was in bed for a first length of time and that the mask was worn by the subject during a second length of time while the subject was in bed (826).

Further, the system determines a PAP adherence metric based on the first length of time and the second length of time (828).

Further, the system causes the PAP adherence metric to be presented to a user (830).

In some implementations, determining the PAP adherence metric can include determining a ratio between the first time and the second time.

In some implementations, the one or more second time intervals can be within a pre-determined range of time. The pre-determined range of time can be selected by at least one of the subject or the user.

In some implementations, the PAP device can be at least one of a continuous positive airway pressure (CPAP) device, a bilevel positive airway pressure (BiPAP) device, or an adaptive servo-ventilation (ASV) device.

In some implementations, the user can be at least one of the subject or a health care provider.

In some implementations, causing the PAP adherence metric to be presented to the user can include generating a graphical user interface using an electronic device. Further, the graphical user interface can indicate the PAP adherence metric.

In some implementations, the method can also include determining whether the PAP adherence metric is less than a threshold value, and selectively presenting a notification to the user based on the determination whether the PAP adherence metric is less than the threshold value.

In some implementations, the method can further include determining that the PAP adherence metric is less than a threshold value, and responsive to determining that the PAP adherence metric is less than the threshold value, presenting a notification to the user.

In some implementations, the method can also include generating, based on the PAP adherence metric, one or more health recommendations for the subject, and causing the one or more health recommendations to be presented to the user.

In some implementations, generating the one or more health recommendations for the subject can include, responsive to determining that the PAP adherence metric is less than the threshold value, generating a first recommendation to the subject to increase a percentage of time that the subject wears the mask while the subject is in bed.

In some implementations, the sensor apparatus can include one or more accelerometers.

In some implementations, the method can further include accessing third data from the sensor apparatus, the second data indicating one or more third time intervals during which the subject was sleeping; determining, based on the third data, that the user was sleeping for a third length of time; and causing the third length of time to be presented to the user.

In some implementations, the method can further include determining a ratio between the third length of time and a target length of time, and causing the ratio between the third length of time and the target length of time to be presented to the user.

In some implementations, the target length of time can be selected based on data representing a sleep pattern of the subject.

In some implementations, the method can include determining whether the third length of time is less than a target length of time, and selectively presenting a notification to the user based on the determination whether the third length of time is less than the target length of time.

In some implementations, the method can further include determining that the third length of time is less than a threshold length of time, and responsive to determining that the third length of time is less than the target length of time, presenting a notification to the user.

In some implementations, the method can further include generating, based on the time length of time, one or more health recommendations for the subject; and causing the one or more health recommendations to be presented to the user.

In some implementations, generating the one or more health recommendations for the subject can include, responsive to determining that the third length of time is less than the threshold length of time, generating a first recommendation to the subject to increase the subject's sleep duration.

In some implementations, generating the one or more health recommendations for the subject can include determining at least one of: a recommended bedtime for the user, or a recommended wake time for the user.

In some implementations, at least one of the recommended bedtime for the user or the recommended wake time for the user can be determined based on data representing a sleep pattern of the subject.

In some implementations, generating the one or more health recommendations for the subject can include generating a first recommendation to the subject to meet a weight loss goal, wherein the first recommendation comprises an indication to increase the subject's sleep duration.

In some implementations, a system can also monitor and track a user's sleep patterns, and provide individualized health recommendations (e.g., sleep hygiene recommendations) to the user based on the monitoring and tracking. As an example, a system can track the amount of time that a user is in bed for sleeping or sleeps each night (e.g., using an activity tracker and/or a sleep tracker), and compare user's sleep time to a recommended amount of time (e.g., 7 to 9 hours a night based on adult healthy sleep recommendations). If the user is below the percent sleep success relative to the recommended sleep, the system can generate a notification to the user (e.g., to notify that the user is not getting enough sleep, and recommend to the user that she sleep more each night).

In some implementations, the user's sleep time can refer to the amount of time that the user is actually sleeping.

In some implementations, the user's sleep time can refer to the amount of time that the user is in bed (e.g., in-bed time), regardless if the user is actually sleeping or is awake). In some implementations, this can be beneficial in providing a meaningful metric to a user in meeting a sleep goal (e.g., a particular duration for healthy sleep), as there may be variability in their actual sleep time (e.g., total sleep duration) occurring over their time spent in bed.

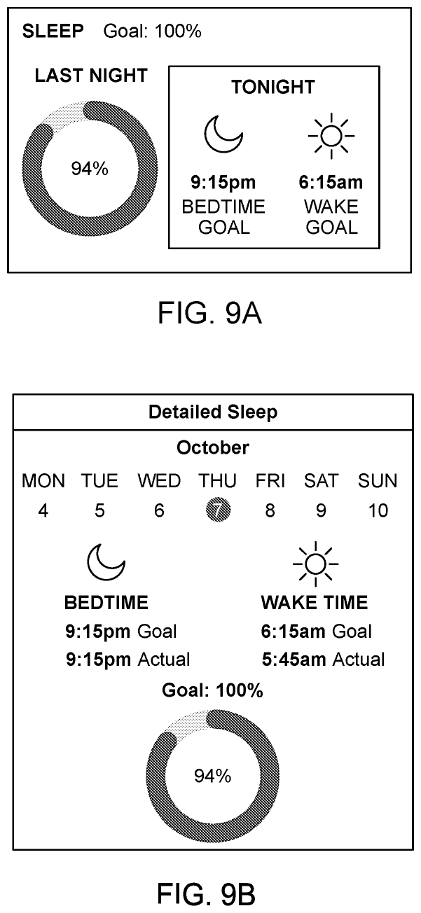

As an example, FIG. 9A show a graphical user interface that can be generated and presented to a user (e.g., using an electronic device, such as a smart phone, smart watch, tablet computer, computer, etc.). In this example, the graphical user interface indicates the amount of that the user slept (or was in bed) the night before compared to the recommended sleep time (or in-bed time). For instance, here the user has a sleep adherence metric of 94% (e.g., the user slept or was in bed 94% of the recommended time). Further, the graphical user interface indicates a recommended bedtime and wake up time that is personalized to the user (e.g., based on their habitual sleep wake habits) to achieve the goal of healthy, recommended sleep time.

As another example, FIG. 9B shows another graphical user interface that can be generated and presented to a user. In this example, the graphical user interface shows detailed information regarding a user's sleeping habits, including a comparison between (i) the recommended bedtime (e.g., a recommended time for the user to go to bed) and recommended wake up time for a particular night, and (ii) the user's actual bedtime and wake up times for that night. Further, the graphical user interface indicates the amount of that the user slept (or was in bed) the night before compared to the recommended sleep time or in-bed time (e.g., in a similar manner as in FIG. 9A, shown as percent sleep metric). The user can select different nights to review detailed sleep information regarding each of those nights.

In some implementations, the recommended sleep time, recommended in-bed time, recommended bedtime, and/or recommended wake up time can be manually specified by the user and/or by a healthcare provider. For example, the user and/or the healthcare provide can determine recommendations based a survey or questionnaire regarding the user's sleep habits, preferences, health condition, etc.

In some implementations, one or more of these recommendations can be automatically determined by a computer system (e.g., by a mobile device, remote server, computer, etc.), such as using a machine learning or artificial intelligence process.

In some implementations, one or more of these recommendations can be individually tailored to the user based on the user's lifestyle and individual needs (e.g., work, family, and/or other personal schedules) and can be check against a minimum threshold amount of time (e.g., 8 hours in bed duration, such as to adhere to healthy sleep guidelines and recommendations by American Academy of Sleep Medicine for adults). In some implementations, one or more of these recommendations can be individually tailored to the user based on the user's age, sleep habits, and/or preference.

In some implementations, if the user meets the recommend sleep time (or in-bed time) the system can determine that the user's sleep adherence metric is 100% (or 1). If the user's less adherence is less than 100% (or 1), the system can add a pre-determined amount of buffer time to the user's sleep time or in-bed time (e.g., 15 minutes or less) and recalculate the user's sleep adherence metric. This can be beneficial, for example, in not discouraging patients if they are nearly (but not completely) adherent to the recommended sleep duration. This can also be beneficial, for example, to account for potential inaccuracies in tracking sleep time and/or in-bed time. In some implementations, the system can refrain from adding a buffer time to the sleep time and/or in bedtime (e.g., such that only the originally determined sleep adherence metric is reported).

In some implementations, the system can track and display information regarding a user's total sleep duration and sleep stages (e.g., deep sleep, REM sleep, etc.), as determined based on sensor data obtained by an activity tracker or sleep tracker.

In some implementations, a system can display portions of the graphical user interface according to different colors and/or patterns in order to indicate different levels of adherence. For example, in the graphical user interfaces shown in FIGS. 9A and 9B, the sleep adherence metric is visually indicated using a donut graph. In some implementations, the color of the donut graph can vary depending on the value of the sleep adherence metric. For example, the donut graph can be red if the sleep adherence metric is less than 75%, yellow if the sleep adherence metric is between 75% and 90%, and green if the sleep adherence metric is greater than 90%. Other colors and/or ranges also can be used, depending on the implementation.

In some implementations, at least some of the sleep monitoring and tracking information described herein can be presented to a healthcare provider (e.g., via a graphical user interface, such as dashboard provided by an application and/or a remote server). The healthcare provider can review the information and input personalized recommendations for the user (e.g., to modify and/or improve their sleeping habits). The recommendations can be provided to the user (e.g., by transmitting the recommendations to the user's device).

In some implementations, a system can automatically generate and present health recommendations to a user based on the sleep monitoring and tracking information. As an example, the system can determine whether a user is sleeping (or is in bed) for the recommendation amount of time, and if not, generate and a present a notification (e.g., personalized sleep hygiene related tasks) to the user to increase their sleep time or in-bed time to a healthier duration.

In some implementations, a system can automatically generate a recommended sleep schedule (or in-bed schedule) for a user, and periodically present the schedule to the user for review. For example, the system can generate a recommended sleep schedule for the next two weeks, and periodically present the schedule for the user over the course of the two weeks so that the user can adhere to the schedule (e.g., in the form of a to do list of check list such as personalized sleep hygiene related tasks). The schedule also can be modified, such as by the user, the health care provider, and/or the system based on the user's sleep patterns, preferences, adherence level, etc. In some implementations, this schedule may be referred to as “Sleep HomeWork.”

Further, the system can generate a notification to a user prior to the recommended bedtime for each particular night (e.g., an hour before the recommended bedtime, or some other specified amount of time beforehand) to remind the user to go adhere to the recommendations or a notification referring to Sleep HomeWork.

In some implementations, the system can also indicate whether the user is adhering to the sleep schedule. For example, if the user has adhered to a particular sleep time for a particular night, the system can display a “congratulations” message to the user (e.g., to reward the user for her efforts). As another example, if the user has not adhered to a particular sleep time for a particular night, the system can display information to a user (e.g., indicating the health benefits of a full night of sleep) to encourage the user to adhere to the recommended schedule. The sleep information can also be analyzed by artificial intelligence systems and algorithms to generate the cross correlations between adherence to sleep and weight loss success and send motivational messages to concurrently improve multiple healthy lifestyle behaviors (e.g., diet, exercise, sleep).

In some implementations, the system can generate health recommendations pertaining to the user's weight loss goals. For example, a user can often achieve weight loss during a combination of diet, exercise, and adequate sleep. Further, in at least some cases, a person's hunger level may increase due to a lack of sleep. Further still, in at least some case, a person may be better able to exercise with a full night of sleep. Accordingly, by obtaining a full night of sleep, a user can both increase their ability to exercise while also reducing their hunger, thereby improving their chances of meeting a weight loss goal. To aid the user in meeting a weight loss goal, the system can generate a recommendation that the user sleep a particular duration each night, a recommendation that user go to bed at a particular time, and/or a recommendation that a user wake up at a particular time (e.g., to achieve 100% adherence to their sleep goals). Further, the system can indicate to the user the weight loss benefits of following these recommendations.

In some implementation, system can also generate a notification requesting that the user provide input regarding how she currently feels (e.g., sleepy, hungry, energetic etc.) after last night's sleep. As an example, the user can provide feedback on a 0 to 100 sliding scale according to one or more metrics, such as sleepiness, energy level, hunger level, etc. This information can be stored and tracked to provide addition contextual information to a health care provider regarding the user's health. This information also can be stored and tracked to reinforce the user's self-motivation behaviors towards behavior change.

As noted above, in some embodiments, the mobile device may maintain one or more application programs, databases, media and/or other information in a main and/or secondary memory. The memory may include, for example, a hard disk drive and/or removable storage drive, representing a floppy disk drive, a magnetic tape drive, an optical disk drive, etc. A removable storage drive may read from and/or write to a removable storage unit in any known manner. The removable storage unit may represent a floppy disk, magnetic tape, optical disk, etc. which is read by and written to by a removable storage drive. As will be appreciated, a removable storage unit may include a computer usable storage medium having stored therein computer software and/or data.

In some embodiments, the mobile device is connected, via a cloud-based platform, to a computer server including a processor coupled to non-transitory, computer-readable memory containing instructions executable by the processor to cause the computer server to receive and analyze the data to generate the score.

In some embodiments, the processor may be provided locally on the mobile device or on a remote server. The processor may be embodied as any type of processor capable of performing the functions described herein. For example, the processor may be embodied as a single or multi-core processor(s), digital signal processor, microcontroller, or other processor or processing/controlling circuit.

In other embodiments, the computer server may be a computer system, computer operated methods, computer products, systems including computer-readable memory, systems including a processor and a tangible, non-transitory memory configured to communicate with the processor, the tangible, non-transitory memory having stored instructions that, in response to execution by the processor, cause the system to perform steps in accordance with the disclosed principles, systems including non-transitory computer-readable storage medium configured to store instructions that when executed cause a processor to follow a process in accordance with the disclosed principles.

In some embodiments, the memory may be random access memory (RAM). The memory may be embodied as any type of device or devices configured for short-term or long-term storage of data such as, for example, memory devices and circuits, memory cards, hard disk drives, solid-state drives, or other data storage devices. Similarly, the memory may be embodied as any type of volatile or non-volatile memory or data storage capable of performing the functions described herein. The computer server may further include one or more application programs directly stored thereon. The application program(s) may include any number of different software application programs, each configured to execute a specific task.

As noted above, in some embodiments, the sleep tracking data are indicative of sleep time or sleep stages. In some embodiments, the sleep tracking data is indicative of time in bed. The methods integrate monitoring treatment adherence and monitoring other health metrics using data obtained from the PAP device as well as data and various health metrics obtained from wearable devices such as a sleep tracker or sleep apnea tracker. For example, in some embodiments, the sleep tracking data are received from a wearable device. Thus, methods disclosed herein have the ability to combine information from multiple devices.

Methods disclosed herein may use the application programming interface (API) for each device to combine data and information from multiple devices. An API is a software intermediary that allows two applications to communicate with each other. For example, the devices may be activity trackers and digital weight scales. Systems disclosed herein may interface with any PAP device, for example any device that includes the sensors, communication channels, processors, and actuators required for generating and transferring PAP usage data. In preferred embodiments, the sleep trackers use 3-axis accelerosensor, heart rate sensors, and/or other sensors (e.g., temperature sensors, respiration sensors, etc.) to reliably determine sleep-wake patterns and time in bed, total sleep duration, and sleep stages. For example, the sleep stages may be one or more of a stage of non-rapid eye movement (NREM) such as NREM Stage 1, NREM Stage 2, or NREM Stage 3 (deep sleep). The sleep stage may be rapid-eye movement sleep (REM) sleep.

In some embodiments, methods disclosed herein include integrated diet, nutrition, activity, and weight-tracking data received and input as health metric data. This data may also be integrated with a companion interventionist web-based dashboard for example a diet, weight, and nutrition tracking dashboard. In some embodiments, the method supports behavioral interventions such as weight-loss or activity coaching or sleep and PAP adherence coaching. The method may include an interventionist and/or coach-facing module to display features related to monitored health metrics including percent PAP adherence. For example, the interventionist and/or coach-facing module may include information related to healthcare team or research studies to which the user belongs and the behaviors targeted for change. The user-facing interface may include features for behavior change interventions, for example to foster weight loss, healthier diet quality, and physical activity, as well as sleep intervention or PAP adherence coaching for encouraging healthier sleep behavior. In this way, the system may present interventionists and users both with relevant information from the PAP adherence tracking platform integrated with diet, activity, weight, and sleep tracking features used to tailor health coaching and intervention to improve health outcomes.

In some embodiments, the data received by the computer server from the PAP device is received via the PAP API via Bluetooth communication. The data may indicate the time the PAP mask was in use during sleep. Further, the sleep tracking data may represent total sleep time. The sleep tracking data may be received via the sleep tracking device API via Bluetooth communication.

In some embodiments, methods disclosed herein further include an automated algorithm, wherein the algorithm determines an overlap between the time the PAP mask is worn and a time the wearer is asleep. an automated algorithm analyzes time in bed and sleep time. The algorithm may first determine the overlap between PAP wear and time in bed using mathematical equations to then express the PAP adherence as percent of time spent in bed. In some embodiments, the computing system is configured to track adherence to treatment over time. This algorithm may also be applied to different sleep stages (e.g., deep sleep and rapid eye movement sleep) captured by wearable device and combined with PAP mask wear during sleep stages. For example, the sleep stages may be one or more of a stage of non-rapid eye movement (NREM) such as NREM Stage 1, NREM Stage 2, or NREM Stage 3 (deep sleep). The sleep stage may be rapid-eye movement sleep (REM) sleep.

In addition, in some embodiments, the methods allow a user to track data associated with one or more health metrics. For example, the health metric may be any metric that affects to sleep patterns such that calculation of the adherence to treatment may be affected by the sleep pattern. For example, the health metric may be one or more of weight, height, age, gender, race/ethnicity, activity, nutrition, socio-economic status, heart rate, prior PAP adherence, apnea-hypopnea index (AHI), BMI, average sleep over time, diet, energy intake, exercise, total energy expenditure, activity energy expenditure, energy balance, resting metabolic rate, weight change, profession, and/or geographical location. In this way, the system includes one or more individualized metrics for calculating the adherence to treatment score.

The health metric may be manually received by the computing system or it may be received through an API of a device. For example, a user's AHI may be determined through a sleep study and then entered into the system for use in determining effectiveness of treatment when the user is in bed or asleep without using the PAP device.

In some embodiments, the methods include tracking nutrition, physical activity, and weight. The interface may display any number of health metrics tracked by the system. Importantly, the system displays the percent PAP adherence each night. This may be by darkening a portion of a circle's circumference to represent the percentage of time the PAP mask was worn during the time spent in bed or asleep. For example, the time the PAP mask was worn may be determined and conveyed by the PAP device via the device API. The time the mask was worn may be determined by the device, for example using pressure-flow sensors, and mask-on time. In embodiments, the sleep tracking data are produced by a wearable device. Thus, the time spent in bed may be conveyed by a wearable device, for example by a wrist-worn activity tracker, activity tracker, and/or sleep tracker worn on another body part such as ring on a finger, or a watch

In some embodiments, the processor may further cause the computing system to send a notification to the PAP wearer via a mobile device such that the notification is a reminder to use the PAP device. In some embodiments, the notification is tailored to habitual bedtimes from the sleep tracker and includes a reminder to use the PAP device and to encourage PAP wear based on individual percent PAP adherence data, thus encouraging and/or improving PAP treatment adherence. For example, the method may further include sending individualized push notifications approximately one hour before the user's bedtime to remind a user to wear the PAP device each night and to sync their sleep-tracker (e.g., a wearable device) each morning upon waking. Notifications may be tailored to the user's self-reported bedtime for the first week and then adjusted to their average bedtime shown by the sleep-tracker as data become available. In some embodiments, the system may also send the wearer encouraging or engaging push notification tailored to the wearer's specific percent PAP adherence and/or sleep adherence from each night. The frequency of the push notifications may be customized by the user and/or the provider. Thus, because the systems and methods disclosed herein provide a more accurate assessment of true adherence, the associated push notifications provide a more meaningful message and feedback to users as compared to prior art systems that simply capture the number of hours the PAP mask is worn.

As noted above, in embodiments, methods disclosed herein may integrate nutrition and weight loss goals with the PAP data. For example, the method may include one or more databases. In some embodiments, the method includes a database to house data produced by the PAP device and the wearable device. The method may further include a same or different database or databases for storing data related to diet and exercise, and for tracking diet and exercise data. Nutrition intake may be tracked when a user searches for and select foods in a database associated with the system or may add custom foods or recipes by recording calorie and fat gram content. Calories and fat gram intake may be totaled each day. Daily calorie and fat gram goals within the nutrition section may be displayed, which are calculated based on the user's weight and/or other health metrics.

Physical activity may be tracked by automatically transferring data from a user's wrist-worn activity tracker (or activity tracker mounted elsewhere on the body) using Bluetooth transfer of data. Physical activities may also be manually entered by searching in the database, which may include a compendium of physical activities rated by their intensity. Activity may be tracked by selecting the specific activity and its duration. Weight may be tracked automatically by syncing with a smart scale via Bluetooth transfer, for example by using a wireless digital weight scale. Weight may also be manually entered into the app if needed. For every health metric tracked and/or monitored by the system, progress over time may be viewed as a weekly and/or monthly line graph.

In some embodiments, the computer server is configured to track a user's percentage of adherence to treatment and to store data in a database.

Reference throughout this specification to “one embodiment” or “an embodiment” means that a particular feature, structure, or characteristic described in connection with the embodiment is included in at least one embodiment. Thus, appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

The term “non-transitory” is to be understood to remove only propagating transitory signals per se from the claim scope and does not relinquish rights to all standard computer-readable media that are not only propagating transitory signals per se. Stated another way, the meaning of the term “non-transitory computer-readable medium” and “non-transitory computer-readable storage medium” should be construed to exclude only those types of transitory computer-readable media which were found in In Re Nuijten to fall outside the scope of patentable subject matter under 35 U.S.C. § 101.

The terms and expressions which have been employed herein are used as terms of description and not of limitation, and there is no intention, in the use of such terms and expressions, of excluding any equivalents of the features shown and described (or portions thereof), and it is recognized that various modifications are possible within the scope of the claims. Accordingly, the claims are intended to cover all such equivalents.

### EXAMPLES

Aspects of the systems and methods disclosed herein are illustrated in the examples provided below.

### Example 1

### Overview

A study was conducted to develop a customized mHealth tool to support treatment adherence to both CPAP and weight loss recommendations in sleep apnea patients.

The goal of the study was to develop a personal treatment adherence and tracking tool combining weight loss features with the percent CPAP adherence tracking (e.g., % CPAP wear time relative to objectively assessed time in bed) for sleep apnea patients.

The study utilized a technology platform for delivering smartphone applications to patients and web-based dashboards to interventionists.

The study included 37 patients aged 10 to 65 years.

### Introduction

Obstructive sleep apnea (OSA) is a common sleep disorder that compounds risk of diabetes and cardiovascular disease in individuals with overweight and obesity.

Continuous positive airway pressure (CPAP), applied at night, is considered the treatment of choice for OSA and there is not FDA-approved drug treatment for OSA. Recommendations to lose weight are also standard in most OSA patients.

Effective OSA treatment requires all-night CPAP use during the entire time spent in bed for optimally treating respiratory events and preventing adverse health effects associated with time spent without wearing CPAP.

The study aimed to develop an improved CPAP adherence module (e.g., a metric that is % CPAP wear time relative to objectively assessed time in bed) that tracks CPAP use as a percent of time spent in bed for sleeping.

### Ethical Considerations

The study was approved by the University of Chicago Institutional Review Board. All participants gave informed consent prior to the study after a member of the research team explained all details of the study, and the subject received satisfactory answers to all of his/her questions.

### Iterative User-Centered Design Process

Through an iterative user-centered design process, the CPAP adherence tracking module of the disclosure was developed, which integrates with a weight loss app with nutrition, activity, weight, and sleep tracking features. This CPAP adherence tracking module was integrated into a mHealth system targeting lifestyle behaviors (nutrition, physical activity) to achieve weight loss in the OSA population.

Adult men and women were recruited with inclusion criteria of age 20 to 65 years and an OSA diagnosis. There were no exclusion criteria based on BMI, race/ethnicity, or other demographic characteristics.

In phase 1 (mean age: 45±8 years), feedback was collected about the weight loss app from patients with known OSA who were receiving CPAP treatment. Participants tested the weight loss app along with connected devices, including wearable devices such as a wrist-worn sleep-activity tracker, and a wireless digital weight scale to self-monitor and receive feedback on their dietary intake, physical activity, and weight for 10 days. At the end of the 10-day period, they completed a survey about the app design which included the System Usability Scale: a measure of usability on a 0 to 100 point scale where 65 is the threshold for a system to be considered usable.

In phase 2 (mean age: 47±9 years), patients with known OSA who were receiving CPAP treatment were studied. Participants completed an online survey to provide their preferences for graphical displays and interpretations of various wireframe images displaying information from the CPAP device tracking module. Using the various metrics of CPAP use that are available via the CPAP device's Application Programming Interface (API), the phase 2 study aimed to develop a customized CPAP adherence tracking module that displayed information deemed the most relevant and helpful by patients who were receiving CPAP treatment. Participants were asked to describe their understanding of the information displayed on each screen (e.g., % adherence [% CPAP wear time relative to objectively assessed time in bed], mask leak, apnea-hypopnea index [AHI, referring to the number of respiratory events per hour of sleep]) and how they would react or change their CPAP use behavior after seeing that particular screen displayed on the app. This phase was done iteratively such that participants' survey responses elicited subsequent modifications in wireframe images, which led to a revised survey design capturing features of the updated wireframe images. A total of four consecutive versions of the CPAP tracking module and survey were produced and presented to participants based on the prior participants' feedback. Thus, during phase 2, participants completed some version of this iterative survey.

In phase 3 (mean age: 55±8 years), patients who were newly-diagnosed with OSA at the University of Chicago Sleep Disorders Clinic and who were CPAP naïve and owned an Android smartphone were recruited following informed consent. This phase involved in-field testing of the CPAP tracking module of the app while participants were using a newly prescribed CPAP machine, a wireless digital weight scale, and a wearable sleep-activity tracker. The app combined the CPAP module with weight loss tracking features so that participants could experience and evaluate the integrated app. The home screen of the CPAP module graphically depicted CPAP adherence as CPAP use relative to the time in bed, expressed as a percentage. The home screen also displayed CPAP use and time in bed separately in hours and minutes. An additional page in the app displayed CPAP adherence over a timeframe of weeks or months, as well as details of daily adherence and mask leak. Each participant was provided an auto-adjusting CPAP machine, wireless digital weight scale, and a wearable sleep-activity tracker to use for 3 to 4 weeks. The wearable sleep-activity data were used to track time spent in bed, and CPAP device API data were used to track CPAP mask wear time to allow calculation of percent CPAP adherence relative to time spent in bed. Participants also received weekly phone calls to troubleshoot any CPAP device, wearable sleep-activity tracker, or smartphone app-related issues.

Additionally, some participants in the later part of the testing received push notifications within an hour of their self-reported bedtime (first week and objectively calculated as the data became available) as a reminder to wear CPAP in the evening. In addition, a message was sent upon the wearable sleep-activity tracker sensing waking to remind the participant to wear and sync the wearable sleep-activity device

In phase 1, patients with known OSA who were receiving CPAP treatment (n=7) tested the weight loss app to track nutrition, activity, and weight for 10 days. Participants completed a usability and acceptability survey. In phase 2, patients with known OSA who were receiving CPAP treatment (n=21) completed an online survey about their interpretations and preferences for wireframes displaying information from the CPAP tracking module. In phase 3, newly-diagnosed OSA patients who were CPAP-naïve (n=9) were prescribed a CPAP machine and tested the app with integrated CPAP and weight loss tracking features for 3 to 4 weeks. Participants completed a usability survey and provided feedback on the app.

During phase 1, participants found the weight loss app mostly easy to use except for some difficulty searching for specific foods in the database. All found the connected devices (wearable activity tracker and wireless electronic scale) easy to use and helpful. During phase 2, participants correctly interpreted CPAP adherence success, expressed as % wear time relative to time in bed and preferred seeing clearly stated “% 100 goal”. In phase 3, participants found the integrated app easy to use and requested push notifications as reminders to wear CPAP before bedtime and to sync the wearable activity tracker in the morning.

### Results

During phase 1, a total of 7 participants (6 men and 1 woman) were enrolled. All participants found the app easy to use except for some difficulty searching for specific foods in the database. All participants endorsed that they would feel confident using the app. No participant found the app too difficult to use, reported that they would need help, or stated that it was overly complex. No participants encountered problems with the connected devices (e.g., wearable activity tracker) or wireless digital weight scales, and they commented that both were “easy to use” and that they liked “how the information from it went straight into the app”. Overall, the participants rated the app an average of 83 on the 100-point System Usability Scale, a score considered to be indicative of a highly usable system compared to other similar systems from a recent review. During phase 2, participants correctly interpreted CPAP adherence success, expressed as % wear time relative to time in bed and expressed a preference for seeing a visual indicator of the CPAP treatment adherence goal. In phase 3, participants found the integrated app easy to use and requested push notifications as reminders to wear CPAP before bedtime and to sync the wearable activity-sleep tracker device in the morning.

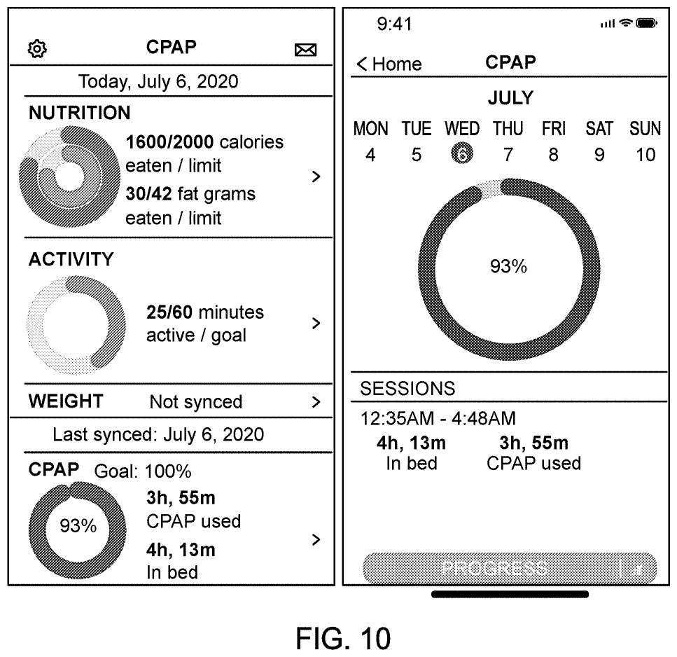

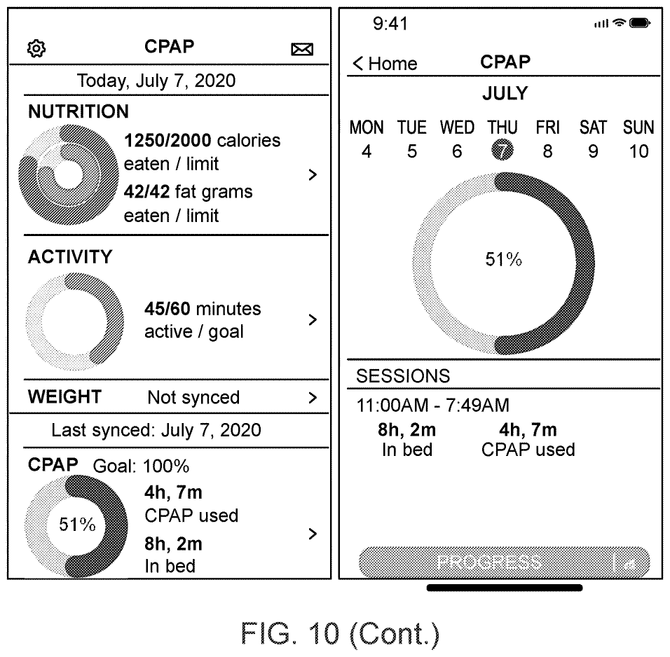

FIG. 10 illustrates an embodiment of the app interfaces. A total of 10 newly diagnosed OSA patients who were CPAP-naïve, were consented. One patient discontinued participation after consent and no data were collected. All 9 participants endorsed that they liked the design, and the app was easy to use and navigate. Of the 6 users who received push notifications, all found them to be helpful and well-timed. Participants rated the app an average of 80 on the 100-point System Usability Scale, a score considered to be indicative of a very usable system. Some participants reported during calls that the app interface did not always show 100% CPAP adherence even when they wore their CPAP mask for the entire time they spent in bed. After carefully examining data in response to this feedback, the percent CPAP adherence calculation logic was adjusted to allow a 15-minute buffer for the time in bed captured by the wearable activity tracker. This minor adjustment in the calculation logic protected against a margin of measurement error for time in bed as captured by the wearable activity tracker, while not compromising the accuracy of the percent CPAP adherence measure.

### Principal Findings

A customized mHealth tool was developed that integrates an improved CPAP adherence tracking module into a weight loss app with diet, activity, and weight tracking. The CPAP adherence tracking module comprises features such as: addressing OSA-obesity comorbidity, CPAP adherence tracking via % CPAP wear time relative to objectively assessed time in bed, and push notifications at meaningful times of day to foster adherence.

Patients were assessed in a three-phase iterative, user-centered process to develop an mHealth tool combining weight loss features with a percent CPAP adherence tracking (e.g., % CPAP wear time relative to objectively measured time in bed) for OSA patients to support both CPAP adherence and weight loss behaviors in patients with OSA and overweight/obesity. The user-centered design process of the systems and methods of the disclosure identified key information that participants find useful for tracking their adherence to CPAP, as well as user interfaces that participants found easy to interpret. Participants were not only satisfied with less information about their CPAP use but also that their interpretations were more accurate when less information was provided. Also, there was a need for explicit descriptions of the information provided, which was accomplished by providing descriptors or comparators such as “Goal: 100%.” The information collected via the surveys supported the design decisions regarding appropriate features, functions, and user interface for the study. Overall, the participants rated the system's usability as highly usable and they reported positive impressions of the app's features. Additionally, based on participants' feedback during phase 3, the usefulness of the app was enhanced by adding push notifications to remind users to wear their CPAP mask (e.g., one hour before bedtime) and wrist activity tracker (e.g., synch device each morning) at meaningful times of the day.

The iterative user-centered design process that was used to develop the app integrating CPAP tracking has notable strengths. A few prior apps designed for OSA populations relied primarily on the views that clinical experts expressed in focus groups with minimal feedback from patients. By engaging patients in the design process from the outset and throughout, the likelihood that the final app will be easy to use, helpful, and engaging to targeted end-users was increased. Low-fidelity wireframes were used to represent user interfaces and embedded them in surveys to gather feedback quickly, without requiring time-consuming programming. The iterative nature of the surveys allowed prompt response to end-user feedback about features or graphical displays that were not functional, not interpreted correctly, or not liked, bringing the development closer to a feasible and acceptable interface.

In this study, the percent CPAP adherence was based on the time spent in bed captured by the wearable activity tracker, and not the actual time spent asleep. CPAP use would ideally be required during the entire sleep period. However, CPAP adherence displayed as a percent of time spent in bed is more meaningful for patients (e.g., end-users or other subjects) in order to meet the goal of CPAP use during all sleep periods occurring over time in bed. Additionally, there is evidence to suggest that specific wearable activity trackers have acceptable levels of measurement accuracy for the time in bed compared to research-grade accelerosensors.

### Incorporation by Reference

References and citations to other documents, such as patents, patent applications, patent publications, journals, books, papers, web contents, have been made throughout this disclosure. All such documents are hereby incorporated herein by reference in their entirety for all purposes.

### Equivalents

Various modifications of the systems and methods of the disclosure and many further embodiments thereof, in addition to those shown and described herein, will become apparent to those skilled in the art from the full contents of this document, including references to the scientific and patent literature cited herein. The subject matter herein contains important information, exemplification and guidance that can be adapted to the practice of systems and methods of this disclosure in its various embodiments and equivalents thereof.

1. A system for treatment adherence monitoring, the system comprising:
a computing system comprising a processor coupled to non-transitory, computer-readable memory containing instructions executable by the processor to cause the computing system to:
receive data from a PAP device associated with use of the device by a wearer;
receive sleep tracking data from a tracking device;
generate, based on an analysis of the data received from the PAP device and the sleep tracking data, a score defining adherence to PAP treatment; and
display the score and data associated with a health metric via an interface on a mobile device.

2. The system of claim 1, wherein the data received from the PAP device is a time a PAP mask was worn by the wearer.

3. The system of claim 2, wherein the sleep tracking data is a time the wearer was in bed for sleeping.

4. The system of claim 3, further comprising an algorithm, wherein the algorithm determines an overlap between the time the PAP mask is worn and a time the wearer is in bed for sleeping.

5. The system of claim 4, wherein the algorithm expresses the adherence to PAP treatment as a percentage based on the time the PAP mask was worn to a total time in bed or sleep duration.

6. The system of claim 2, wherein the sleep tracking data is a time the wearer was in bed for sleeping, a total sleep duration, and a time the wearer was in a sleep stage.

7. The system of claim 1, wherein the data associated with a health metric comprises one or more of weight, height, age, gender, race/ethnicity, BMI, activity, nutrition, socio-economic status, profession, geographical location, heart rate, prior PAP adherence, measures of the severity of sleep apnea, apnea-hypopnea index (AHI), oxygen desaturation index (ODI), average sleep over time, diet, energy intake, exercise, energy expenditure metrics, energy balance, resting metabolic rate, or weight change.

8. The system of claim 1, wherein the score and the data associated with a health metric are presented as feedback to a user.

9. The system of claim 1, wherein the sleep tracking data are produced by a wearable device.

10. The system of claim 1, wherein the data associated with a health metric are produced by a wearable device.

11. The system of claim 10, wherein the wearable device is a sleep apnea sensor.

12. The system of claim 1, wherein the processor further causes the computing system to send a notification to the wearer via the mobile device, wherein the notification is a reminder to use the PAP device.

13. The system of claim 12, wherein the notification is based on data received for the wearer over a period of time.

14. The system of claim 1, wherein the computing system is configured to track percent adherence to treatment over time.

15. The system of claim 1, wherein the processor is provided locally on the mobile device or provided on a remote server.

16. The system of claim 1, wherein the computing system is configured to communicate and exchange data over a network.

17. A method for providing treatment adherence monitoring in a subject, the method comprising:
receiving data from a PAP device associated with a use of the device by a wearer;
receiving, from one or more different devices, sleep tracking data;
generating, based on an analysis of the data received from the PAP device and the sleep tracking data, a score defining a percentage of an adherence to PAP treatment; and
displaying the score and data associated with a health metric via an interface on a mobile device.

18. The method of claim 17, wherein the mobile device is connected, via a cloud-based platform, to a computer server comprising a processor coupled to non-transitory, computer-readable memory containing instructions executable by the processor to cause the computer server to receive and analyze the data to generate the score.

19. The method of claim 17, wherein the data received from the PAP device is a time a PAP mask was worn by the wearer.

20. The method of claim 19, wherein the sleep tracking data is a time the wearer was asleep.

21. The method of claim 20, further comprising an algorithm, wherein the algorithm determines an overlap between the time the PAP mask is worn and a time the wearer is in bed.

22. The method of claim 21, wherein the algorithm expresses the adherence to PAP treatment as a percentage based on the time the PAP mask was worn to the time the wearer was asleep.

23. The method of claim 17, wherein the sleep tracking data is a time the wearer was in bed for sleeping, a total sleep duration, and a time the wearer was in a sleep stage.

24. The method of claim 17, wherein the data associated with a health metric comprises one or more of weight, height, age, gender, race/ethnicity, BMI, activity, nutrition, socio-economic status, profession, geographical location, heart rate, prior PAP adherence, measures of the severity of sleep apnea, apnea-hypopnea index (AHI), oxygen desaturation index (ODI), average sleep over time, diet, energy intake, exercise, energy expenditure metrics, energy balance, resting metabolic rate, or weight change

25. The method of claim 17, wherein the score and the data associated with a health metric are presented as feedback to encourage and/or improve PAP treatment adherence.

26. The method of claim 17, wherein the sleep tracking data is received from a sleep-wake monitor.

27. The method of claim 17, wherein the processor further causes the computer server to send a notification to the wearer via the mobile device, wherein the notification comprises a reminder to use the PAP device.

28. The method of claim 27, wherein the notification is based on data received for the wearer over a continuous period of time.

29. The method of claim 17, wherein the computer server is configured to track a wearer's percentage of adherence to treatment and the data associated with a health metric over time, wherein tracking comprises storing the data in a database.

30. A method comprising:
accessing, by one or more processors, first data from a positive airway pressure (PAP) device, the first data indicating one or more first time intervals during which a mask of the PAP device was worn by a subject;
accessing, by the one or more processors, second data from a sensor apparatus worn by the subject, the second data indicating one or more second time intervals during which the subject was in bed;
determining, by the one or more processors based on the first data and the second data, that the subject was in bed for a first length of time and that the mask was worn by the subject during a second length of time while the subject was in bed;
determining, by the one or more processors, a PAP adherence metric based on the first length of time and the second length of time; and
causing, by the one or more processors, the PAP adherence metric to be presented to a user.

31. The method of claim 30, wherein determining the PAP adherence metric comprises determining a ratio between the first time and the second time.

32. The method of claim 30, wherein the one or more second time intervals are within a pre-determined range of time, wherein the pre-determined range of time is selected by at least one of the subject or the user.

33. The method of claim 30, wherein the PAP device is at least one of a continuous positive airway pressure (CPAP) device, a bilevel positive airway pressure (BiPAP) device, or an adaptive servo-ventilation (ASV) device.

34. The method of claim 30, wherein the user is at least one of the subject or a health care provider.

35. The method of claim 30, wherein causing the PAP adherence metric to be presented to the user comprises generating a graphical user interface using an electronic device, wherein the graphical user interface indicates the PAP adherence metric.

36. The method of claim 30, further comprising:
determining whether the PAP adherence metric is less than a threshold value, and
selectively presenting a notification to the user based on the determination whether the PAP adherence metric is less than the threshold value.

37. The method of claim 36, further comprising:
determining that the PAP adherence metric is less than a threshold value, and
responsive to determining that the PAP adherence metric is less than the threshold value, presenting a notification to the user.

38. The method of claim 37, further comprising:
generating, based on the PAP adherence metric, one or more health recommendations for the subject; and
causing the one or more health recommendations to be presented to the user.

39. The method of claim 38, wherein generating the one or more health recommendations for the subject comprise:
responsive to determining that the PAP adherence metric is less than the threshold value, generating a first recommendation to the subject to increase a percentage of time that the subject wears the mask while the subject is in bed.

40. The method of claim 30, wherein the sensor apparatus comprises one or more accelerometers.

41. The method of claim 30, further comprising:
accessing third data from the sensor apparatus, the second data indicating one or more third time intervals during which the subject was sleeping;
determining, based on the third data, that the user was sleeping for a third length of time; and
causing the third length of time to be presented to the user.

42. The method of claim 41, further comprising:
determining a ratio between the third length of time and a target length of time, and
causing the ratio between the third length of time and the target length of time to be presented to the user.

43. The method of claim 42, wherein the target length of time is selected based on data representing a sleep pattern of the subject.

44. The method of claim 41, further comprising:
determining whether the third length of time is less than a target length of time, and
selectively presenting a notification to the user based on the determination whether the third length of time is less than the target length of time.

45. The method of claim 41, further comprising:
determining that the third length of time is less than a threshold length of time, and
responsive to determining that the third length of time is less than the target length of time, presenting a notification to the user.

46. The method of claim 41, further comprising:
generating, based on the time length of time, one or more health recommendations for the subject; and
causing the one or more health recommendations to be presented to the user.

47. The method of claim 46, wherein generating the one or more health recommendations for the subject comprises:
responsive to determining that the third length of time is less than the threshold length of time, generating a first recommendation to the subject to increase the subject's sleep duration.

48. The method of claim 46, wherein generating the one or more health recommendations for the subject comprises:
determining at least one of:
a recommended bedtime for the user, or
a recommended wake time for the user.

49. The method of claim 48, wherein at least one of the recommended bedtime for the user or the recommended wake time for the user is determined based on data representing a sleep pattern of the subject.

50. The method of claim 46, wherein generating the one or more health recommendations for the subject comprises:
generating a first recommendation to the subject to meet a weight loss goal, wherein the first recommendation comprises an indication to increase the subject's sleep duration.

51. A system comprising:
a positive airway pressure (PAP) device comprising a mask configured to be worn by a subject;
a sensor apparatus configured to be worn by the subject, the sensor apparatus comprising an accelerometer; and
an electronic device communicatively coupled to the PAP device and the sensor apparatus, wherein the electronic device is configured to:
access first data from the PAP device, the first data indicating one or more first time intervals during which the mask was worn by the subject;
accessing second data from the sensor apparatus, the second data indicating one or more second time intervals during which the subject was in bed;
determining, based on the first data and the second data, that the subject was in bed for a first length of time and that the mask was worn by the subject during a second length of time while the subject was in bed;
determining a PAP adherence metric based on the first length of time and the second length of time; and
causing the PAP adherence metric to be presented to a user.

52. The system of claim 51, wherein determining the PAP adherence metric comprises determining a ratio between the first time and the second time.

53. The system of claim 51, wherein the one or more second time intervals are within a pre-determined range of time, wherein the pre-determined range of time is selected by at least one of the subject or the user.

54. The system of claim 51, wherein the PAP device is at least one of a continuous positive airway pressure (CPAP) device, a bilevel positive airway pressure (BiPAP) device, or an adaptive servo-ventilation (ASV) device.

55. The system of claim 51, wherein the user is at least one of the subject or a health care provider.

56. The system of claim 51, wherein causing the PAP adherence metric to be presented to the user comprises generating a graphical user interface using an electronic device, wherein the graphical user interface indicates the PAP adherence metric.

57. The system of claim 51, wherein the electronic device is further configured to:
determine whether the PAP adherence metric is less than a threshold value, and
selectively present a notification to the user based on the determination whether the PAP adherence metric is less than the threshold value.

58. The system of claim 57, wherein the electronic device is further configured to:
determine that the PAP adherence metric is less than a threshold value, and
responsive to determining that the PAP adherence metric is less than the threshold value, present a notification to the user.

59. The system of claim 58, wherein the electronic device is further configured to:
generate, based on the PAP adherence metric, one or more health recommendations for the subject; and
cause the one or more health recommendations to be presented to the user.

60. The system of claim 59, wherein generating the one or more health recommendations for the subject comprise:
responsive to determining that the PAP adherence metric is less than the threshold value, generating a first recommendation to the subject to increase a percentage of time that the subject wears the mask while the subject is in bed.

61. The system of claim 51, wherein the sensor apparatus comprises one or more accelerometers.

62. The system of claim 51, wherein the electronic device is further configured to:
access third data from the sensor apparatus, the second data indicating one or more third time intervals during which the subject was sleeping;
determine, based on the third data, that the user was sleeping for a third length of time; and
cause the third length of time to be presented to the user.

63. The system of claim 62, wherein the electronic device is further configured to:
determine a ratio between the third length of time and a target length of time, and
cause the ratio between the third length of time and the target length of time to be presented to the user.

64. The system of claim 63, wherein the target length of time is selected based on data representing a sleep pattern of the subject.

65. The system of claim 61, wherein the electronic device is further configured to:
determine whether the third length of time is less than a target length of time, and
selectively present a notification to the user based on the determination whether the third length of time is less than the target length of time.

66. The system of claim 61, wherein the electronic device is further configured to:
determine that the third length of time is less than a threshold length of time, and
responsive to determining that the third length of time is less than the target length of time, present a notification to the user.

67. The system of claim 61, wherein the electronic device is further configured to:
generate, based on the time length of time, one or more health recommendations for the subject; and
cause the one or more health recommendations to be presented to the user.

68. The system of claim 67, wherein generating the one or more health recommendations for the subject comprises:
responsive to determining that the third length of time is less than the threshold length of time, generating a first recommendation to the subject to increase the subject's sleep duration.

69. The system of claim 67, wherein generating the one or more health recommendations for the subject comprises:
determining at least one of:
a recommended bedtime for the user, or
a recommended wake time for the user.

70. The system of claim 69, wherein at least one of the recommended bedtime for the user or the recommended wake time for the user is determined based on data representing a sleep pattern of the subject.

71. The system of claim 67, wherein generating the one or more health recommendations for the subject comprises:
generating a first recommendation to the subject to meet a weight loss goal, wherein the first recommendation comprises an indication to increase the subject's sleep duration.
